(19) United States
(12) Patent Application Publication
(10) Pub. No.: US 2017/0209415 A1
(43) Pub. Date: Jul. 27, 2017

(54) USE OF (+)-1-(3,4-DICHLOROPHENYL)-3-AZABICYCLO[3.1.0]HEXANE TO TREAT ADDICTIVE DISORDERS INCLUDING NICOTINE ADDICTION

(71) Applicants: Anthony Alexander McKinney, Cambridge, MA (US); Franklin Bymaster, Brownsburg, IN (US); Phil Skolnick, Edgewater, NJ (US)

(72) Inventors: Anthony Alexander McKinney, Cambridge, MA (US); Franklin Bymaster, Brownsburg, IN (US); Phil Skolnick, Edgewater, NJ (US)

(21) Appl. No.: 15/257,756

(22) Filed: Sep. 6, 2016

Related U.S. Application Data
(63) Continuation of application No. 13/964,033, filed on Aug. 9, 2013, now abandoned.
(60) Provisional application No. 61/682,314, filed on Aug. 13, 2012.

(51) Int. Cl. A61K 31/403; A61K 9/20; A61K 9/48; A61K 45/06

(52) U.S. Cl. CPC A61K 31/403; A61K 45/06; A61K 9/20; A61K 9/48

(57) ABSTRACT

The present invention relates to (+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane and their use alone or in combination with additional anti-addictive compositions in the treatment of nicotine addiction and related disorders.

This application claims priority to U.S. Provisional Application No. 61/682,314, filed Aug. 13, 2012, the contents of which are incorporated herein by reference.

### TECHNICAL FIELD

The present invention relates to the treatment of addiction. Specifically, the present invention relates to the use of (+)-1-(3,4-dichlorophenyl)-3-azabicyclo[3.1.0]hexane or pharmaceutically acceptable salts, polymorphs, glycosylated derivatives, metabolites, solvates, hydrates, and/or prodrugs of (+)-1-(3,4-dichlorophenyl)-3-azabicyclo[3.1.0]hexane and their use in the treatment or prevention of addictive disorders, particularly nicotine addiction.

### ADDITIONAL DISCLOSURE

This application includes the additional disclosure of U.S. patent application Ser. No. 13/310,694, filed Dec. 2, 2011, U.S. Provisional patent application Ser. No. 61/662,462, filed Jun. 21, 2012, U.S. Provisional patent application Ser. No. 61/677,453, filed Jul. 30, 2012, U.S. Provisional patent application Ser. No. 61/573,499, filed Sep. 6, 2011, U.S. Continuation patent application Ser. No. 13/366,209, filed Feb. 3, 2012, U.S. patent application Ser. No. 13/335,981, filed Dec. 23, 2011, U.S. patent application Ser. No. 10/466,457, filed Jan. 11, 2002, now U.S. Pat. No. 7,098,229, U.S. patent application Ser. No. 09/753,883, filed Jan. 11, 2011, now U.S. Pat. No. 6,372,919, U.S. Continuation patent application Ser. No. 13/297,452, filed Nov. 16, 2011, and U.S. Continuation patent application Ser. No. 13/366,211, filed Feb. 3, 2012, each of which are incorporated herein by reference in their entirety for all purposes.

### BACKGROUND OF THE INVENTION

Addiction to nicotine, alcohol, and pharmaceutical agents represents a significant public health concern at a cost upwards of half a trillion dollars a year in the United States alone (The Science of Addiction, NIH Pub. No 10-5605, August, 2010), with drugs and alcohol estimated to contribute to the death of more than 100,000 people and tobacco estimated to contribute to the death of more than 440,000 people annually (The Science of Addiction, NIH Pub. No 10-5605, August, 2010). Additionally, those that use addictive substances, particularly nicotine, frequently have a higher incidence of depressive disorders and are at risk for depression or depressive episodes when attempting to stop such behaviors (Glassman et al., 1990).

Substances that trigger dependencies in human beings increase the release of dopamine in the nucleus accumbens. (Di Chiara et al., 2004). However, not all substances that trigger dependencies increase the release of dopamine in the same way. For example, some substances such as morphine and nicotine immitate natural neuromediators. Other substances such as cocaine increase the secretion of natural neuromediators, while others such as alcohol block neuromediators (Dubuc, 2002). Addiction has also been linked to monoaminergic transmitters including norepinephrine and serotonin (Majchrowicz, 1973; Li et al., 1998; Luscher et al., 2006; Benowitz and Peng 2000; Dudas and George 2005; Frishman 2007; Rezvani et al. 1990).

Treatment strategies for addiction have tried to: directly target the dopamine transporter with analogs, inhibit reuptake of dopamine, modulate synaptic dopamine directly through the use of dopamine agonists or antagonists, or modulate synaptic dopamine by specifically targeting a functionally linked but biochemically different neurotransmitter system. However, treatment of addiction remains an issue. An agonist may act on more than one receptor and may create a dependency for the agonist. Substitution therapy such as nicotine substitution therapy involves the administration of the addictive substance in a different form, frequently leading to withdrawal and subsequent relapse. There is therefore a need for alternative treatments for addiction, particularly nicotine addiction.

1-(3,4-dichlorophenyl)-3-azabicyclo[3.1.0]hexane is a triple reuptake inhibitor that was initially described in U.S. Pat. No. 4,231,935 and U.S. Pat. No. 4,196,120 as a non-narcotic analgesic. Amitifadine, (1R,5S)-(+)-1-(3,4-dichlorophenyl)-3-azabicyclo[3.1.0]hexane or (+)-1-(3,4-dichlorophenyl)-3-azabicyclo[3.1.0]hexane of Formula I, below,

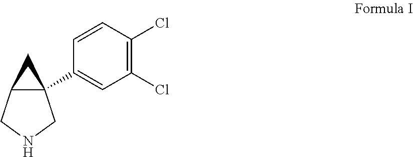 [SMILES: Clc1ccc([C@]23CNC[C@H]2C3)cc1Cl]

is one of the enantiomers of 1-(3,4-dichlorophenyl)-3-azabicyclo[3.1.0]hexane and was described as an anti-depressant in U.S. Pat. No. 7,098,229.

Enantiomers may have the same or different effects on biological entities and many pharmaceutical agents are sold as racemates even though the desired or any pharmacological activity resides in only one enantiomer. For example, the S(+)-methacholine enantiomer is 250 times more potent than the R(−) enantiomer. With ketamine, the (S)-enantiomer is an anesthetic, but the (R)-enantiomer is a hallucinogen. Administration of a racemic mixture of any drug can be disadvantageous in that racemic mixtures may be less pharmacologically active than one of the enantiomers as in the case of methacholine, or it may have increased toxicity or other undesirable side effects as in ketamine.

While the current array of drug treatments for nicotine addiction are more effective than placebo, they fall far short of being effective for the majority of users (Frishman et al., 2006). There therefore remains a need for effective treatment of addiction to nicotine.

### SUMMARY OF EXEMPLARY EMBODIMENTS

Provided herein are compositions and methods using an unbalanced triple reuptake inhibitor, amitifadine, (+)-1-(3,4-dichlorophenyl)-3-azabicyclo[3.1.0]hexane, as shown below, and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane in the treatment and prevention of addiction in mammalian animals, including humans. Such addictive disorders include, but are not limited to, addiction to or abuse of substances such as nicotine. Additionally provided herein are means for reducing or eliminating withdrawal effects from mammalian animals stopping or attempting to stop addictive behaviors.

Unbalanced as used herein refers to the relative effects on each of the monoamine transporters. In this case reference is made to a triple reuptake inhibitor with the most activity against the serotonin transporter, half as much to the norepinephrine transporter, and one eighth to the dopamine transporter. In contrast, a balanced triple reuptake inhibitor would have similar activity against each of the three monoamine transporters.

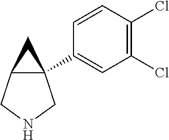 [SMILES: Clc1ccc([C@]23CNC[C@H]2C3)cc1Cl]

### Amitifadine, (+)-1-(3,4-dichlorophenyl)-3-azabicyclo[3.1.0]hexane

(+)-1-(3,4-dichlorophenyl)-3-azabicyclo[3.1.0]hexane agents as used herein are substantially free of the corresponding (−) enantiomer, (+1-(3,4-dichlorophenyl)-3-azabicyclo[3.1.0]hexane. In addition to being enantiomeric, (+)-1-(3,4-dichlorophenyl)-3-azabicyclo[3.1.0]hexane exists in at least three polymorphic forms, labeled herein polymorphs A, B and C. The polymorphs may be used in pharmaceutical compositions in combination or in forms that are substantially free of one or more of the other polymorphic forms.

(+)-1-(3,4-dichlorophenyl)-3-azabicyclo[3.1.0]hexane may furthermore be in the form of pharmaceutically acceptable active salts, glycosylated derivatives, metabolites, solvates, hydrates and/or prodrugs. For example, many pharmacologically active organic compounds regularly crystallize incorporating second, foreign molecules, especially solvent molecules, into the crystal structure of the principal pharmacologically active compound to form pseudopolymorphs. When the second molecule is a solvent molecule, the pseudopolymorphs can also be referred to as solvates. Additionally, pharmaceutically acceptable forms may include inorganic and organic acid addition salts such as hydrochloride salt.

Additional background information pertaining to (+)-1-(3,4-dichlorophenyl)-3-azabicyclo[3.1.0]hexane may be found, for example, in U.S. Pat. No. 6,372,919, U.S. Pat. No. 7,098,229, U.S. patent application Ser. No. 11/205,956, U.S. patent application Ser. No. 11/493,431, U.S. patent application Ser. No. 11/740,667, U.S. patent application Ser. No. 11/936,016, U.S. patent application Ser. No. 12/135,053, U.S. patent application Ser. No. 12/208,284, U.S. patent application Ser. No. 12/334,432, U.S. patent application Ser. No. 12/428,399, U.S. patent application Ser. No. 12/782,705, U.S. patent application Ser. No. 12/895,788, U.S. patent application Ser. No. 13/048,852, U.S. Provisional Patent Application No. 61/419,769, WO/20040466457, WO2007127396, WO02066427, WO2006023659, each of which is incorporated herein by reference in their entirety. Additional information pertaining to (+)-1-(3,4-dichlorophenyl)-3-azabicyclo[3.1.0]hexane and the treatment of addiction and addictive behaviors may be found in U.S. patent application Ser. No. 13/310,694, filed Dec. 2, 2011, and its predecessor application, U.S. Provisional Patent Application No. 61/419,769, filed Dec. 3, 2010, each incorporated by reference herein in their entirety.

Additionally provided herein are combinatorial compositions and coordinate treatment means using additional or secondary psychotherapeutic agents in combination with (+)-1-(3,4-dichlorophenyl)-3-azabicyclo[3.1.0]hexane agents including (+)-1-(3,4-dichlorophenyl)-3-azabicyclo[3.1.0]hexane, and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane. Suitable secondary psychotherapeutic drugs for use in the treatment and prevention of addiction in the compositions and methods herein include, but are not limited to, drugs from the general classes of anti-convulsants, mood-stabilizing, anti-psychotic, anxiolytic, opioid receptor antagonist, aldehyde dehydrogenase inhibitor, calcium channel blockers, nicotinic receptor desensitizing agents, al receptor binding agents, and antidepressants. (See, e.g., R J. Baldessarini in Goodman & Gilman's The Pharmacological Basis of Therapeutics, 11th Edition, Chapters 17 and 18, McGraw-Hill, 2005 for a review). Exemplary secondary psychotherapeutic drugs for use in the compositions and methods herein include, but are not limited to, 3-propoxy-β-carboline hydrochloride, naltrexone, acamprosate, disulfiram, topirmate, bupropion and varenicline.

It is shown herein that use of (+)-1-(3,4-dichlorophenyl)-3-azabicyclo[3.1.0]hexane agents ((+)-1-(3,4-dichlorophenyl)-3-azabicyclo[3.1.0]hexane, and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) are effective in treating, preventing, alleviating, or moderating addictions affected by monoamine neurotransmitters or biogenic amines, specifically addictions that are alleviated by inhibiting dopamine and/or norepinephrine and/or serotonin reuptake including, but not limited to, addictive or substance abuse disorders such as addiction to abuse of nicotine.

It is additionally shown herein that the use of (+)-1-(3,4-dichlorophenyl)-3-azabicyclo[3.1.0]hexane agents ((+)-1-(3,4-dichlorophenyl)-3-azabicyclo[3.1.0]hexane, and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) is effective in preventing, alleviating, or decreasing withdrawal effects from cessation of the use of addictive substances or disorders including, but not limited to, addictive or substance abuse disorders such as addiction to abuse of nicotine.

It is further shown herein that the use of (+)-1-(3,4-dichlorophenyl)-3-azabicyclo[3.1.0]hexane agents ((+)-1-(3,4-dichlorophenyl)-3-azabicyclo[3.1.0]hexane, and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) is effective in preventing, alleviating, or decreasing depression caused or triggered by cessation of the use of addictive substances or disorders including, but not limited to, addictive or substance abuse disorders such as addiction to or abuse of nicotine.

The unbalanced serotonin-norepinephrine-dopamine reuptake inhibition ratio of ˜1:2:8, respectively, of (+)-1-(3,4-dichlorophenyl)-3-azabicyclo[3.1.0]hexane (Skolnick et al., 2003) allows for higher dosages of (+)-1-(3,4-dichlorophenyl)-3-azabicyclo[3.1.0]hexane to be used without triggering the dopaminergic or norepinephrine side effects such as elevated heart rate, increased blood pressure, gastrointestinal (nausea/vomiting and constipation/diarrhea) effects, dry mouth, insomnia, anxiety, and hypomania seen in similar dosages of balanced triple reuptake inhibitors or unbalanced triple reuptake inhibitors with different inhibition ratios.

The effectiveness of amitifadine, (+)-1-(3,4-dichlorophenyl)-3-azabicyclo[3.1.0]hexane in the treatment of addiction, particularly nicotine addiction, is surprising given that amitifadine more strongly inhibits serotonin and norepinephrine reuptake than dopamine reuptake. Administration of pharmaceutical compositions comprising (+)-1-(3,4-dichlorophenyl)-3-azabicyclo[3.1.0]hexane agents including (+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane in effective amounts will be effective to decrease cravings for addictive substances, improving an individual's score on one or more multidimensional scales such as Questionnaire of Smoking Urges (QSU) developed by Tiffany & Drobes (Br. J. Addict. 86(11):1467-76 (1991)), Measurement of Drug Craving scale (Sayette et al. 2000), Drug History Questionnaire (DHQ), Desires for Drug Questionnaire (Franken et al., Addict. Behav. 27:675-85 (2002)), Heaviness of Smoking Index (HSI), the Fagerstrom Test for Nicotine Dependence (FTND) or the Obsessive-Compulsive Beliefs Questionnaire-87 (OBQ-87).

In accordance with this invention, a dosage form has been developed for the sustained or extended release delivery of an active ingredient of (+)-1-(3,4-dichlorophenyl)-3-azabicyclo[3.1.0]hexane agents including (+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane in effective amounts to treat addictions for a long period of time. In one exemplary embodiment, the active ingredient can be administered in an effective amount to provide sustained plasma levels and activity within the methods of the invention by utilizing a dosage regimen of from about 25 mg to about 200 mg once or twice daily in an oral unit dosage composition containing the active ingredient, 30% to 50% by weight of the composition of a pharmaceutically acceptable carrier, and from about 15% to 45% by weight of the composition of a hydroxypropyl methyl cellulose slow release matrix.

The present invention may be understood more fully by reference to the detailed description and examples which are intended to exemplify non-limiting embodiments of the invention.

### BRIEF DESCRIPTION OF THE DRAWINGS

FIG. 1 is a series of graphs showing that over ten days there is a significant increase in horizontal activity in a 90 minute open field locomotor test in alcohol preferring (P) rats administered 0.4 mg/kg nicotine in comparison to non-alcohol preferring (NP) rats administered the same amount of nicotine.

FIG. 2 shows that (+)-1-(3,4-dichlorophenyl)-3-azabicyclo[3.1.0]hexane (DOV 21,947) in P rats significantly reduced nicotine-mediated increases in open field locomotor activity (A) and that (+)-1-(3,4-dichlorophenyl)-3-azabicyclo[3.1.0]hexane alone did not significantly alter open field locomotor activity (B). * P<0.001 by analysis of variance between groups (ANOVA).

FIG. 3 is a series of graphs showing that P rats exhibit an enhanced sensitivity to the reward potentiating effects of nicotine in the ICSS paradigm following sensitization in comparison to NP rats as measured by rate frequency function of P vs. NP rats when tested on a 300-20 HZ descending frequency schedule (A), minimum frequency, EF₅₀ and maximum frequency (B) and total responding (C). * P<0.001 by ANOVA.

FIG. 4 is a series of graphs showing the response of P rats to varying amounts of nicotine using the ICSS paradigm as measured by (A) Rate frequency function of P rats when tested on a 300-20 Hz descending frequency schedule with multiple doses of nicotine; (B) Minimum frequency; (C) EF50; and (D) total responding. * P<0.001 by ANOVA.

FIG. 5 shows the effect of amitifadine ((+)-1-(3, 4-dichlorophenyl)-3-azabicyclo[3.1.0]hexane) directly injected into the medial prefrontal cortex (10, 20, and 40 μg) on the nicotine facilitation of ICSS as evidenced by a left-ward shift from PBS in the rate frequency function tested on P rats over a 300-20 Hz descending frequency schedule (A); Minimum frequency, EF50 and maximum frequency parameters (B); and total responding (C). * P<0.001 by ANOVA.

FIG. 6 shows time-dependent elevations in the threshold of P rats using the ICSS paradigm following nicotine withdrawal as measured by time-dependent increase of minimum frequency (A); EF50 (B); and decreases in total responding (C). * P<0.001 by ANOVA.

FIG. 7 shows the effect of amitifadine ((+)-1-(3, 4-dichlorophenyl)-3-azabicyclo[3.1.0]hexane) on ICSS during 48 hour nicotine withdrawal as measured by minimum frequency, EF50 (A) and total responding (B) at 48 hours of nicotine withdrawal. * P<0.001 by ANOVA.

FIG. 8 shows the effects of nicotine withdrawal at 48 hours after the last dose of nicotine on P rats in a forced swim test paradigm without (A) and with amitifadine ((+)-1-(3, 4-dichlorophenyl)-3-azabicyclo[3.1.0]hexane) (B). * P<0.001 by ANOVA. P rats were sensitized by administering nicotine (0.4 mg/kg sc) for 14 days, and then nicotine was withdrawn over a period of 48 or 72 hours.

FIG. 9 is a graph showing the mean effect of various concentrations of (+)-1-(3,4-dichlorophenyl)-3-azabicyclo[3.1.0]hexane (amitifadine) on nicotine self-administration averaged over two test phases (mean±standard error of the mean (sem)). *=P<0.05 and **=P<0.005 versus control.

FIG. 10 is a graph showing the effect of 0, 5 (p<0.025), 10 (p<0.0005) or 30 (p<0.0005) mg/kg of amitifadine ((+)-1-(3,4-dichlorophenyl)-3-azabicyclo[3.1.0]hexane) on nicotine self-administration during 15-minute time block of a 45-minute session (mean±sem). *=P<0.025, **=P<0.005.

FIG. 11 is two charts showing the effect of various concentrations of (+)-1-(3,4-dichlorophenyl)-3-azabicyclo[3.1.0]hexane (amitifadine) on nicotine self-administration in phases 1 (p<0.05) (A) and 2 (B) (p<0.005).

### DETAILED DESCRIPTION OF EXEMPLARY EMBODIMENTS

Described herein is amitifadine, an enantiomer of (±)-1-(3,4-dichlorophenyl)-3-azabicyclo[3.1.0]hexane, which provides therapeutic efficacy in the treatment of conditions affected by monoamine neurotransmitters including, but not limited to, addiction and addictive disorders including addiction to or abuse of substances such as nicotine. The enantiomer of (±)-1-(3,4-dichlorophenyl)-3-azabicyclo[3.1.0]hexane additionally provides relief from withdrawal symptoms caused by cessation of consumption of addictive substances such as nicotine and addictive behaviors such as smoking. Using the methods of the invention, such disorders are amenable to treatment, prophylaxis, and/or alleviation of the disorder and/or associated symptom(s) such as withdrawal by inhibiting reuptake of multiple biogenic amines causally linked to the targeted disorder. Further described herein are coordinate treatment methods and combined drug compositions, dosage forms, packages, and kits for preventing or treating conditions affected by monoamine neurotransmitters including, but not limited to, addiction.

(+)-1-(3,4-dichlorophenyl)-3-azabicyclo[3.1.0]hexane is a triple reuptake inhibitor (TRI), or serotonin-norepinephrine-dopamine reuptake inhibitor (SNDRI). It was previously described in U.S. Pat. No. 6,372,919.

(+)-1-(3,4-dichlorophenyl)-3-azabicyclo[3.1.0]hexane possesses a desirable unbalanced triple monoamine uptake inhibition ratio with highly potent serotonin reuptake inhibition and lesser norepinephrine and, particularly, dopamine reuptake inhibition in a ratio of ˜1:2:8, respectively (IC50 values of 12, 23, and 96 nM, respectively in human embryonic kidney (HEK) 293 cells expressing the corresponding human recombinant transporters for [3H]serotonin, [3H]norepinephrine, and [3H]dopamine). (Skolnick et al., 2003). An unbalanced triple reuptake inhibitor may provide a lower side effect profile than a balanced triple reuptake inhibitor and allow for higher concentrations of an unbalanced inhibitor such as (+)-1-(3,4-dichlorophenyl)-3-azabicyclo[3.1.0]hexane to be used without incurring the dopaminergic and/or noradrenergic side effects frequently seen in the use of balanced triple reuptake inhibitors or unbalanced triple reuptake inhibitors that have different inhibition ratios.

Provided herein are compositions and methods using the (+) enantiomer of (±)-1-(3,4-dichlorophenyl)-3-azabicyclo[3.1.0]hexane, amitifadine, as shown below,

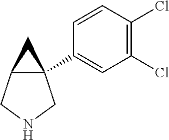 [SMILES: Clc1ccc([C@]23CNC[C@H]2C3)cc1Cl]

(+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane, for the treatment of mammals, including humans, suffering from signs and symptoms of disorders generally treated with triple reuptake inhibitors including, but not limited to, addiction, specifically nicotine addiction. As shown herein, the triple reuptake inhibitor (+)-1-(3,4-dichlorophenyl)-3-azabicyclo[3.1.0]hexane was efficacious in treating nicotine consumption in rat and mouse models. Given the well-developed association of the dopamine transporter with nicotine-related and addictive disorders, the efficacy of (+)-1-(3,4-dichlorophenyl)-3-azabicyclo[3.1.0]hexane for treating nicotine consumption was surprising since this compound more strongly inhibits serotonin and norepinephrine reuptake than dopamine reuptake.

An efficient means of preparing (+)-1-(3,4-dichlorophenyl)-3-azabicyclo[3.1.0]hexane is described in U.S. patent application Ser. No. 11/740,667, incorporated herein by reference in its entirety. Additional exemplary means of preparing (+)-1-(3,4-dichlorophenyl)-3-azabicyclo[3.1.0]hexane may be found, for example, in U.S. patent application Ser. Nos. 10/920,748, 11/205,956; 12/208,284; 12/428,399, WO20040466457, WO2007127396, WO02066427, WO2006023659, and U.S. Pat. No. 6,372,919, each of which is incorporated herein by reference in its entirety.

Methods for preparing (±)-1-(3,4-dichlorophenyl)-3-azabicyclo[3.1.0]hexane may be found, for example, in U.S. Pat. No. 4,435,419 and U.S. patent application Ser. Nos. 10/920,748, 11/205,956; 12/208,284; 12/428,399 each of which is incorporated herein by reference in their entirety

As used herein, the term “substantially pure (+)-1-(3,4-dichlorophenyl)-3-azabicyclo[3.1.0]hexane” or “enantiomerically pure (+)-1-(3,4-dichlorophenyl)-3-azabicyclo[3.1.0]hexane” means that the compositions have been enriched to contain substantially more (+)-1-(3,4-dichlorophenyl)-3-azabicyclo[3.1.0]hexane than (−)-1-(3,4-dichlorophenyl)-3-azabicyclo[3.1.0]hexane. In exemplary embodiments, the compositions exhibit an enantiomeric excess of the (+)-enantiomer that is greater than 80% ee, preferably greater than 90% ee, more preferably greater than 95% ee, and in some cases 98% ee or more enantiomerically enriched for the (+)-1-(3,4-dichlorophenyl)-3-azabicyclo[3.1.0]hexane enantiomer, e.g., as determined by configuration and/or optical activity. Typically, the substantially pure (+)-1-(3,4-dichlorophenyl)-3-azabicyclo[3.1.0]hexane enantiomer composition will contain no more than about 5% w/w of the corresponding (−) enantiomer, more preferably no more than about 2%, more preferably no more than about 1% w/w of the corresponding (−)-1-(3,4-dichlorophenyl)-3-azabicyclo[3.1.0]hexane enantiomer.

In addition to being an unpredictably active, enantiomeric form of 1-(3,4-dichlorophenyl)-3-azabicyclo[3.1.0]hexane, (+)-1-(3,4-dichlorophenyl)-3-azabicyclo[3.1.0]hexane is polymorphic. In this context, the present invention includes compositions and methods employing one or more polymorphic forms of (+)-1-(3,4-dichlorophenyl)-3-azabicyclo[3.1.0]hexane, specifically one of more of polymorphic forms A, B and C, e.g., as disclosed in U.S. patent application Ser. Nos. 11/205,956, 12/208,284 and 12/428,399 each of which is incorporated herein by reference in its entirety.

Polymorph form A may be described as the hemi-hydrate of acid addition salts of (+)-1-(3,4-dichlorophenyl)-3-azabicyclo[3.1.0]hexane. The polymorphs of acid addition salts of (+)-1-(3,4-dichlorophenyl)-3-azabicyclo[3.1.0]hexane may be characterized by their X-ray powder diffraction patterns (XRPD) and/or their Raman spectroscopy peaks. A Bragg-Brentano instrument, which includes the Shimadzu system, used for the X-ray powder diffraction pattern measurements reported herein, gives a systematic peak shift (all peaks can be shifted at a given “° 2θ” angle) which result from sample preparation errors as described in Chen et al.; J Pharmaceutical and Biomedical Analysis, 2001; 26, 63. Therefore, any “° 2θ” angle reading of a peak value is subject to an error of about (±) 0.2°.

The following Table 1 shows the values for the relative intensities for peaks of the X-ray powder diffraction pattern of purified polymorph form A of the hydrochloride salt of (+)-1-(3,4-dichlorophenyl)-3-azabicyclo[3.1.0]hexane having a crystal size of from about 10 to 40 microns. With respect to the percent value of relative intensity (I/Io) given in Table 1, Io represents the value of the maximum peak determined by XRPD for the sample for all “° 2θ” angles and I represents the value for the intensity of a peak measured at a given “° 2θ” angle”. The angle “° 2θ” is a diffraction angle which is the angle between the incident X-rays and the diffracted X-rays.

TABLE 1
XRPD Peaks (°2θ) and Relative Intensities (I/Io) for Polymorph Form A
Form A

| °2θ | I/Io |
|---|---|
| 4.55 | 25 |
| 9.10 | 15 |
| 13.65 | 6 |
| 17.14 | 60 |
| 17.85 | 11 |
| 18.24 | 23 |
| 18.49 | 14 |
| 19.27 | 14 |
| 19.62 | 22 |
| 21.74 | 15 |
| 21.96 | 100 |
| 22.24 | 12 |
| 23.01 | 7 |
| 24.52 | 43 |
| 24.79 | 10 |
| 26.74 | 52 |
| 27.44 | 11 |
| 27.63 | 17 |
| 28.36 | 16 |
| 28.48 | 26 |
| 29.00 | 14 |
| 29.20 | 19 |
| 29.40 | 27 |
| 29.57 | 27 |
| 30.24 | 18 |
| 31.01 | 13 |
| 31.62 | 17 |
| 32.20 | 24 |
| 32.93 | 12 |
| 33.42 | 9 |
| 34.24 | 6 |
| 35.08 | 15 |
| 35.65 | 16 |
| 36.31 | 14 |
| 37.11 | 26 |
| 37.78 | 9 |
| 39.85 | 9 |

The following Table 2 shows the relative intensities for peaks of the X-ray powder diffraction pattern of purified polymorph form B of the hydrochloride salt of (+)-1-(3,4-dichlorophenyl)-3-azabicyclo[3.1.0]hexane having a crystal size of from about 10 to 40 microns.

TABLE 2
XRPD Peaks (°2θ) and Relative Intensities (I/Io) for Polymorph Form B
Form B

| °2θ | I/Io |
|---|---|
| 10.50 | 6 |
| 13.34 | 12 |
| 15.58 | 42 |
| 17.12 | 6 |
| 17.36 | 8 |
| 17.52 | 26 |
| 18.21 | 11 |
| 20.40 | 7 |
| 21.35 | 97 |
| 21.61 | 17 |
| 21.93 | 11 |
| 22.64 | 6 |
| 23.04 | 79 |
| 24.09 | 6 |
| 24.52 | 14 |
| 25.43 | 96 |
| 26.24 | 53 |
| 26.36 | 73 |
| 26.75 | 11 |
| 26.88 | 7 |
| 27.44 | 6 |
| 27.94 | 12 |
| 28.36 | 20 |
| 28.54 | 30 |
| 29.39 | 10 |
| 29.72 | 9 |
| 30.07 | 7 |
| 30.58 | 8 |
| 30.72 | 100 |
| 31.07 | 14 |
| 31.38 | 12 |
| 31.55 | 7 |
| 31.78 | 12 |
| 32.14 | 10 |
| 32.31 | 7 |
| 32.80 | 7 |
| 32.95 | 6 |
| 33.45 | 44 |
| 33.74 | 12 |
| 35.25 | 10 |
| 35.40 | 12 |
| 35.58 | 9 |
| 36.75 | 8 |
| 37.55 | 18 |
| 39.01 | 15 |
| 39.22 | 7 |
| 39.37 | 7 |
| 39.86 | 11 |

The following Table 3 shows the values of the relative intensities of the peaks of the X-ray powder diffraction pattern of purified polymorph form C of the hydrochloride salt of (+)-1-(3,4-dichlorophenyl)-3-azabicyclo[3.1.0]hexane having a crystal size of from about 10 to 40 microns.

TABLE 3
XRPD Peaks (°2θ) and Relative Intensities (I/Io) for Polymorph Form C
Form C

| °2θ | I/Io |
|---|---|
| 5.46 | 6 |
| 5.66 | 20 |
| 6.37 | 6 |
| 7.26 | 6 |
| 8.75 | 6 |
| 13.34 | 25 |
| 13.94 | 11 |
| 15.65 | 7 |
| 16.26 | 7 |
| 17.01 | 8 |
| 17.38 | 9 |
| 17.64 | 83 |
| 17.92 | 15 |
| 18.23 | 40 |
| 19.08 | 7 |
| 19.38 | 46 |
| 19.86 | 20 |
| 20.07 | 100 |
| 21.16 | 17 |
| 21.32 | 94 |
| 21.64 | 37 |
| 22.42 | 25 |
| 22.70 | 12 |
| 22.97 | 70 |
| 23.31 | 6 |
| 24.09 | 15 |
| 24.86 | 94 |
| 25.24 | 32 |
| 25.38 | 49 |
| 26.12 | 13 |
| 26.32 | 90 |
| 26.87 | 18 |
| 27.21 | 39 |
| 27.90 | 54 |
| 28.14 | 8 |
| 28.56 | 32 |
| 28.74 | 17 |
| 29.20 | 6 |
| 29.72 | 6 |
| 29.92 | 26 |
| 30.54 | 13 |
| 30.72 | 19 |
| 30.96 | 31 |
| 31.42 | 7 |
| 31.68 | 11 |
| 31.80 | 15 |
| 31.97 | 6 |
| 32.43 | 21 |
| 33.26 | 12 |
| 33.40 | 15 |
| 33.64 | 25 |
| 33.84 | 18 |
| 34.11 | 15 |
| 34.70 | 11 |
| 35.07 | 8 |
| 35.64 | 11 |
| 35.91 | 8 |
| 36.09 | 21 |
| 37.80 | 12 |
| 38.06 | 6 |
| 38.17 | 6 |
| 39.04 | 6 |
| 39.23 | 8 |
| 39.77 | 7 |

There are key major peaks at given angles in these X-ray powder diffraction patterns which are unique to each given polymorph form. These peaks are present in the XRPD patterns of each of the polymorph forms having a crystal size of about 10 to 40 microns. Any of these major peaks, either alone or in any distinguishing combination, are sufficient to distinguish one of the polymorph forms from the other two polymorph forms. For polymorph form A, the “° 2θ” angles of these major peaks which characterize polymorph form A, subject to the error set forth above, are as follows: 17.14; 19.62; 21.96; 24.52; and 26.74. For polymorph form B, the “° 2θ” angles of these major peaks which characterize polymorph form B, subject to the error set forth above, are as follows: 15.58; 17.52; 21.35; 23.04; 25.43; and 30.72. For polymorph form C, the “° 2θ” angles of these major peaks which characterize polymorph form C, subject to the error set forth above, are as follows: 13.34; 17.64; 20.07; 21.32; 22.97; 24.86; 26.32; and 27.90. Any of these major peaks, either alone or in any distinguishing combination, are sufficient to distinguish a polymorph from the other polymorph forms.

Another method of characterizing the three polymorphs of (+)-1-(3,4-dichlorophenyl)-3-azabicyclo[3.1.0]hexane is through Raman spectroscopy. The procedure for carrying out Raman Spectroscopy is described on pages 260-275 of Skoog and West, Principles of Instrumental Analysis (2nd Ed.); Saunders College, Philadelphia (1980).

The Raman spectra peak positions in wavenumbers (cm⁻¹) for polymorph form A, B and C of the hydrochloride salt of (+)-1-(3,4-dichlorophenyl)-3-azabicyclo[3.1.0]hexane are given in Table 4, below.

TABLE 4
Raman Peak Listing for Polymorph Form A, B and C (peaks >
400 cm⁻¹) Peak Positions In Wavenumbers (cm⁻¹)

| Form A | Form B | Form C | | Form B | Form A |
|---|---|---|---|---|---|
| 436 | 418 | 441 | 1246 | 1245 | 1135 |
| 479 | 446 | 474 | 1266 | 1278 | 1189 |
| 534 | 478 | 532 | 1279 | 1309 | 1229 |
| 549 | 533 | 648 | 1309 | 1343 | 1274 |
| 646 | 648 | 674 | 1343 | 1380 | 1309 |
| 691 | 676 | 690 | 1398 | 1398 | 1338 |
| 680 | 686 | 767 | 1456 | 1456 | 1366 |
| 762 | 767 | 811 | 1471 | 1483 | 1393 |
| 812 | 825 | 826 | 1557 | 1557 | 1453 |
| 836 | 852 | 856 | 1595 | 1593 | 1484 |
| 892 | 895 | 895 | 2900 | 2895 | 1557 |
| 921 | 964 | 970 | 2966 | 2963 | 1597 |
| 959 | 979 | 1031 | 2992 | 2993 | 2890 |
| 982 | 1031 | 1059 | 3048 | 3027 | 2969 |
| 998 | 1054 | 1094 | 3070 | 3066 | 2982 |
| 1030 | 1070 | 1122 | | | 3017 |
| 1056 | 1099 | 1137 | | | 3046 |
| 1099 | 1136 | 1189 | | | 3064 |
| 1122 | 1189 | 1228 | | | |

Table 4 provides the complete patterns of the Raman peak positions with respect to the hydrochloride salts of polymorph forms A, B and C respectively. However, there are certain key peaks within these patterns which are unique to each of the hydrochloride salts of these polymorphs. Any of these key peaks, either alone or in any distinguishing combination, are sufficient to distinguish one of the polymorph forms from the other two polymorph forms. These peak positions, expressed in wavenumbers (cm⁻¹) for the hydrochloride salt of polymorph form A are: 762; 836; 921; 959; 1393; 1597; 2890; 2982; and 3064. The characterizing peak positions expressed in wavenumbers (cm⁻¹) for the hydrochloride salt of polymorph form B are: 1245; 1380; 2963; 2993; 3027; and 3066. The characterizing peak positions expressed in wavenumbers (cm⁻¹) for the hydrochloride salt of polymorph form C are: 1059; 1094; 1266; 1343; 1595; 2900; 2966; and 3070. Any of these key peaks, either alone or in any distinguishing combination, are sufficient to distinguish each polymorph form from the other two polymorph forms.

Polymorph forms A, B and C of (+)-1-(3,4-dichlorophenyl)-3-azabicyclo[3.1.0]hexane, particularly as hydrochloride acid addition salts, can each be prepared substantially free of its other enantiomeric, geometric and polymorphic isomeric forms through re-crystallization of a mixture of the A and B polymorph forms produced in accordance with prior art procedures. Depending upon the particular solvent, conditions and concentrations of materials utilized to re-crystallize the mixture of polymorph forms A and B, one can selectively produce the desired polymorph form of (+)-1-(3,4-dichlorophenyl)-3-azabicyclo[3.1.0]hexane, substantially free of its other enantiomeric, geometric and polymorphic isomers. The term “substantially free” of its other enantiomeric, geometric and polymorphic isomeric forms designates that the crystalline material is at least about 95% by weight pure in that it contains no more than about 5% w/w of its other enantiomeric, geometric and polymorphic isomeric forms.

In preparing polymorph forms A and B substantially free of other polymorph forms, crystallization from a mixture of A and B may be utilized. However, the crystallization technique with regard to producing each of these polymorph forms substantially free of other polymorph forms is different. In preparing polymorph form A, which is the hemi-hydrate of the acid addition salt of (+)-1-(3,4-dichlorophenyl)-3-azabicyclo[3.1.0]hexane, it is best to utilize a solvent medium to dissolve a solid containing polymorph form A such as a mixture of polymorph forms A and B in an organic solvent which contains water. The preferred organic solvents that can be utilized in this procedure include lower alkanol solvents such as methanol, butanol, ethanol or isopropanol as well as other solvents such as acetone, dichloromethane and tetrahydrofuran.

Polymorph form B is the anhydrous form of the acid addition salt of (+)-1-(3,4-dichlorophenyl)-3-azabicyclo[3.1.0]hexane. Polymorph form B of the acid addition salt of (+)-1-(3,4-dichlorophenyl)-3-azabicyclo[3.1.0]hexane can be prepared from a solid containing polymorph form A or a mixture of polymorph forms A and B by dissolving the polymorph form A or the mixture of polymorph forms A and B, preferably as the hydrochloride salt, utilizing anhydrous conditions.

Polymorph form C can be prepared from either polymorph form A or polymorph form B or mixtures thereof. Polymorph form C is prepared by extensive heating of either polymorph form A or polymorph form B, or mixtures thereof, at temperatures of at least 50° C., preferably from 60° C. to 80° C. Heating can be continued until polymorph form C substantially free of other polymorph forms is formed.

The techniques set forth above also allow for the preparation of mixtures of the individual polymorph forms of the acid addition salt of (+)-1-(3,4-dichlorophenyl)-3-azabicyclo[3.1.0]hexane containing specific amounts of each of the polymorphs. In particular, mixtures of polymorph form A and either polymorph form B or polymorph form C; polymorph form B and polymorph form C; and polymorph form A, polymorph form B and polymorph form C can be readily prepared with the desired amounts of each of the polymorphs. Using the techniques set forth above, mixtures containing specific percentages of the individual polymorphic forms of the acid addition salt of (+)-1-(3,4-dichlorophenyl)-3-azabicyclo[3.1.0]hexane can be obtained. For example, mixtures containing from about 10% to about 10-20%, 20-35%, 35-50%, 50-70%, 70-85%, 85-95% and up to 95-99% or greater (by weight) of polymorph form A, with the remainder of the mixture being either or both polymorph form B and polymorph form C, can be prepared. As another example, mixtures containing from about 10% to about 10-20%, 20-35%, 35-50%, 50-70%, 70-85%, 85-95% and up to 95-99% or greater (by weight) of polymorph form B, with the remainder of the mixture being either or both polymorph form A and polymorph form C, can be prepared. As a further example, mixtures containing from about 10% to about 10-20%, 20-35%, 35-50%, 50-70%, 70-85%, 85-95% and up to 95-99% or greater (by weight) of polymorph form C, with the remainder of the mixture being either or both polymorph form A and polymorph form B, can be prepared.

Additionally, many pharmacologically active organic compounds regularly crystallize incorporating second, foreign molecules, especially solvent molecules, into the crystal structure of the principal pharmacologically active compound to form pseudopolymorphs. When the second molecule is a solvent molecule, the pseudopolymorphs can also be referred to as solvates. All of these additional forms of (+)-1-(3,4-dichlorophenyl)-3-azabicyclo[3.1.0]hexane are likewise contemplated for use within the present invention.

The polymorph forms A, B and C of the present invention can be prepared as acid addition salts formed from an acid and the basic nitrogen group of (+)-1-(3,4-dichlorophenyl)-3-azabicyclo[3.1.0]hexane. Suitable acid addition salts are formed from acids, which form non-toxic salts, examples of which are hydrochloride, hydrobromide, hydroiodide, sulphate, hydrogen sulphate, nitrate, phosphate, and hydrogen phosphate. Examples of pharmaceutically acceptable addition salts include inorganic and organic acid addition salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; organic acid salts such as acetate, citrate, lactate, succinate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate, tartrate, gluconate and the like. The hydrochloride salt formed with hydrochloric acid is an exemplary useful salt.

As disclosed herein, (+)-1-(3,4-dichlorophenyl)-3-azabicyclo[3.1.0]hexane agents ((+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) are effective in treating a variety of conditions including, but not limited to, addiction. Within related aspects of the invention, combinatorial formulations are provided that use substantially pure (+)-1-(3,4-dichlorophenyl)-3-azabicyclo[3.1.0]hexane, or pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane alone or in combination with other psychotherapeutic or anti-addictive drugs to modulate, prevent, alleviate, ameliorate, reduce or treat symptoms or conditions influenced by monoamine neurotransmitters or biogenic amines. Subjects amenable to treatment according to the invention include mammalian subjects, including humans, suffering from or at risk for any of a variety of conditions including, addiction such as, but not limited to, addiction to nicotine.

(+)-1-(3,4-dichlorophenyl)-3-azabicyclo[3.1.0]hexane or a pharmaceutically acceptable active salt, polymorph, glycosylated derivative, metabolite, solvate, hydrate, and/or prodrug of (+)-1-(3,4-dichlorophenyl)-3-azabicyclo[3.1.0]hexane may be administered alone or in combination with one or more other psychotherapeutic drugs including, but not limited to, drugs from the general classes of anti-convulsants, mood-stabilizing, anti-psychotic, anxiolytic, opioid receptor antagonist, aldehyde dehydrogenase inhibitor, calcium channel blockers, nicotinic receptor desensitizing agents, al receptor binding agents, and antidepressants. (See, e.g., R J. Baldessarini in Goodman & Gilman's The Pharmacological Basis of Therapeutics, 11th Edition, Chapters 17 and 18, McGraw-Hill, 2005 for a review). Exemplary secondary psychotherapeutic drugs for use in the compositions and methods herein include, but are not limited to, 3-propoxy-β-carboline hydrochloride (3-PBC), naltrexone, acamprosate, disulfiram, topirmate, bupropion and varenicline.

Within the coordinate administration methods of the invention, the secondary therapeutic and/or psychotherapeutic drug is administered concurrently or sequentially with (+)-1-(3,4-dichlorophenyl)-3-azabicyclo[3.1.0]hexane, or a pharmaceutically acceptable active salt, polymorph, glycosylated derivative, metabolite, solvate, hydrate, and/or prodrug of (+)-1-(3,4-dichlorophenyl)-3-azabicyclo[3.1.0]hexane to treat or prevent one or more symptoms of the targeted disorder. When administered simultaneously, the additional therapeutic and/or psychotherapeutic agent and (+)-1-(3,4-dichlorophenyl)-3-azabicyclo[3.1.0]hexane or a pharmaceutically acceptable active salt, polymorph, glycosylated derivative, metabolite, solvate, hydrate, and/or prodrug of (+)-1-(3,4-dichlorophenyl)-3-azabicyclo[3.1.0]hexane may be combined in a single composition or combined dosage form. Alternatively, the combinatorially effective additional therapeutic and/or psychotherapeutic drug and (+)-1-(3,4-dichlorophenyl)-3-azabicyclo[3.1.0]hexane agents (including pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) may be administered at the same time in separate dosage forms. When the coordinate administration is conducted simultaneously or sequentially, the additional therapeutic and/or psychotherapeutic agent and (+)-1-(3,4-dichlorophenyl)-3-azabicyclo[3.1.0]hexane agent may each exert biological activities and therapeutic effects over different time periods, although a distinguishing aspect of all coordinate treatment methods of the invention is that treated subjects exhibit positive therapeutic benefits.

Administration of (+)-1-(3,4-dichlorophenyl)-3-azabicyclo[3.1.0]hexane, a pharmaceutically acceptable active salt, polymorph, glycosylated derivative, metabolite, solvate, hydrate, and/or prodrug of (+)-1-(3,4-dichlorophenyl)-3-azabicyclo[3.1.0]hexane or the coordinate treatment method or combinatorial drug composition of the invention to suitable subjects will yield a reduction in one or more target symptom(s) associated with the selected disorder or development of the disorder by at least 2%, 5%, 10%, 20%, 30%, 50% or greater, up to a 75-90%, or 95% or greater, compared to placebo-treated or other suitable control subjects. Comparable levels of efficacy are contemplated for the entire range of disorders described herein, including all contemplated neurological and psychiatric disorders, and related conditions and symptoms, for treatment or prevention using the compositions and methods of the invention. These values for efficacy may be determined by comparing accepted therapeutic indices or clinical values for particular test and control individuals over a course of treatment/study, or more typically by comparing accepted therapeutic indices or clinical values between test and control groups of individuals using standard human clinical trial design and implementation.

As used herein, the terms “prevention” and “preventing,” when referring to a disorder or symptom, refers to a reduction in the risk or likelihood that a mammalian subject will develop said disorder, symptom, condition, or indicator after treatment according to the invention, or a reduction in the risk or likelihood that a mammalian subject will exhibit a recurrence or relapse of said disorder, symptom, condition, or indicator once a subject has been treated according to the invention and cured or restored to a normal state. In one embodiment, (+)-1-(3,4-dichlorophenyl)-3-azabicyclo[3.1.0]hexane or a pharmaceutically acceptable salt thereof is administered as a preventative measure to a patient. According to this embodiment, the patient can have a genetic predisposition to an addictive disorder alleviated by inhibiting dopamine reuptake, such as a family history of such a disorder, or a non-genetic predisposition to an addictive disorder.

In accordance with the invention, compounds disclosed herein, optionally formulated with additional ingredients in a pharmaceutically acceptable composition, are administered to mammalian subjects, for example a human patient, to treat or prevent one or more symptom(s) of a disorder alleviated by inhibiting dopamine reuptake, and/or norepinephrine reuptake, and/or serotonin reuptake. In certain embodiments, “treatment” or “treating” refers to amelioration of one or more symptom(s) of a disorder, whereby the symptom(s) is/are alleviated by inhibiting dopamine and/or norepinephrine and/or serotonin reuptake. In other embodiments, “treatment” or “treating” refers to an amelioration of at least one measurable physical parameter associated with addiction such as craving and/or withdrawal. In yet another embodiment, “treatment” or “treating” refers to inhibiting or reducing the progression or severity of a disorder (or one or more symptom(s) thereof) alleviated by inhibiting dopamine and/or norepinephrine and/or serotonin reuptake, e.g., as discerned based on physical, physiological, and/or psychological parameters. In additional embodiments, “treatment” or “treating” refers to delaying the onset of a disorder (or one or more symptom(s) thereof) alleviated by inhibiting dopamine and/or norepinephrine and/or serotonin reuptake, i.e. delaying the onset of craving or withdrawal symptoms or physical manifestations of such conditions.

An “effective amount,” “therapeutic amount,” “therapeutically effective amount,” or “effective dose” of (+)-1-(3,4-dichlorophenyl)-3-azabicyclo[3.1.0]hexane agent (including pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) and/or an additional psychotherapeutic agent as used herein means an effective amount or dose of the active compound as described herein sufficient to elicit a desired pharmacological or therapeutic effect in a human subject. In the case of anti-addictive therapeutic agents, these terms most often refer to a measurable, statistically significant reduction in an occurrence, frequency, or severity of one or more symptom(s) of a specified disorder, including any combination of neurological and/or psychological symptoms, diseases, or conditions, associated with or caused by the targeted disorder and/or reduction in the development of addiction in a target population.

Therapeutic efficacy can alternatively be demonstrated by a decrease in the frequency or severity of symptoms associated with the treated condition or disorder, or by altering the nature, occurrence, recurrence, or duration of symptoms associated with the treated condition or disorder. In this context, “effective amounts,” “therapeutic amounts,” “therapeutically effective amounts,” and “effective doses” of additional psychotherapeutic drugs and (+)-1-(3,4-dichlorophenyl)-3-azabicyclo[3.1.0]hexane agents (including pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) within the invention can be readily determined by ordinarily skilled artisans following the teachings of this disclosure and employing tools and methods generally known in the art, often based on routine clinical or patient-specific factors.

An unbalanced triple reuptake inhibitor such as (+)-1-(3,4-dichlorophenyl)-3-azabicyclo[3.1.0]hexane may provide a lower side effect profile than a balanced triple reuptake inhibitor and allow for higher concentrations of the unbalanced inhibitor to be used without incurring the dopaminergic and/or noradrenergic side effects frequently seen in the use of balanced triple reuptake inhibitors such as GSK372475 or unbalanced triple reuptake inhibitors that have different inhibition ratios such as SEP-22589.

In contrast to GSK372475, (+)-1-(3,4-dichlorophenyl)-3-azabicyclo[3.1.0]hexane is well tolerated and has a similar adverse event profile as placebo. (+)-1-(3,4-dichlorophenyl)-3-azabicyclo[3.1.0]hexane use also did not lead to the noradrenergic side effects such as significantly elevated heart rate and increased systolic and diastolic blood pressure seen with GSK37425 or dopaminergic side effects such as nausea, vomiting, and hypomania (See, U.S. patent application Ser. No. 13/310,694, filed Dec. 2, 2011, and Graff, et al. 2009).

The SEP-22589 inhibition profile for 5-HT, NE and DA (IC₅₀'s, SEP-289: 15, 4 and 3 nM (Schrieber, 2009)) is about equipotent for norepinephrine and dopamine reuptake inhibition and less potent for serotonin reuptake inhibition, leading to higher rates of noradrenergic or dopaminergic side effects than similar anti-depressant effective amounts of (+)-1-(3,4-dichlorophenyl)-3-azabicyclo[3.1.0]hexane.

The use of (+)-1-(3,4-dichlorophenyl)-3-azabicyclo[3.1.0]hexane will have substantially fewer dopaminergic or noradrenergic side effects than use of similar doses of balanced triple reuptake inhibitors. The use of substantially pure (+)-1-(3,4-dichlorophenyl)-3-azabicyclo[3.1.0]hexane will reduce adverse effects including side effects by 1%, 3%, 10%, 20%, 30%, 50% or greater, up to a 75%, 80%, 90%, or 95% or greater over use of a balanced triple reuptake inhibitor. Additionally, the use of (+)-1-(3,4-dichlorophenyl)-3-azabicyclo[3.1.0]hexane will have fewer dopaminergic or noradrenergic side effects than triple reuptake inhibitors with higher rates of inhibition for dopamine or noradrenaline reuptake. Thus, the use of substantially pure (+)-1-(3,4-dichlorophenyl)-3-azabicyclo[3.1.0]hexane will allow relatively greater reuptake inhibition of the 5-HT (serotonin) transporter, less of the NE (norepinephrine) transporter and even less of the DA (dopamine) transporter which allows maximal improvement of psychiatric symptoms while reducing adverse dopaminergic or noradrenergic effects including side effects by 1%, 3%, 10%, 20%, 30%, 50% or greater, up to a 75%, 80%, 90%, or 95% or greater over use of unbalanced triple reuptake inhibitors with higher rates of inhibition for dopamine or noradrenaline reuptake inhibitors.

The use of (+)-1-(3,4-dichlorophenyl)-3-azabicyclo[3.1.0]hexane will result in reuptake inhibition of the 5-HT transporter in individuals of about 10%, 15%, 20%, 30%, 50% or greater, up to a 75%, 80%, 90%, or 95% or greater than reuptake inhibition of the NE transporter or the DA transporter. In some embodiments, reuptake inhibition of the 5-HT transporter will be two, three, four, five, six, seven or eight fold greater than the reuptake inhibition of the DA transporter. In other embodiments, reuptake inhibition of the 5-HT transporter will be one and half or twice that of the NE transporter. Reuptake inhibition of the NE transporter may be about 10%, 15%, 20%, 30%, 50% or greater, up to a 75%, 80%, 90%, or 95% or greater than reuptake inhibition of the DA transporter. In some embodiments, reuptake inhibition of the NE transporter may be two, three or four times greater than the reuptake inhibition of the DA transporter.

The use of (+)-1-(3,4-dichlorophenyl)-3-azabicyclo[3.1.0]hexane will result in binding of the 5-HT transporter in individuals at levels of about 10%, 15%, 20%, 30%, 50% or greater, up to a 75%, 80%, 90%, or 95% or greater than binding of the NE transporter or the DA transporter. In some embodiments, binding of the 5-HT transporter will be more than about 100% greater than the binding of the NE transporter or the DA transporter. In some embodiments, binding of the 5-HT transporter will be two, three, four, five, six, seven or eight fold greater than the binding of the DA transporter. In other embodiments, binding of the 5-HT transporter will be one and half or twice that of the NE transporter. Binding of the NE transporter may be about 10%, 15%, 20%, 30%, 50% or greater, up to a 75%, 80%, 90%, or 95% or greater than binding of the DA transporter in treated individuals. In some embodiments, binding of the NE transporter may be two, three or four times greater than binding of the DA transporter in an individual. The relative binding as determined by Kᵢ of 5-HT may be slightly higher, substantially higher, or significantly higher than the binding of the DA transporter or NE transporter alone or in combination.

(+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane are useful for treating or preventing endogenous disorders alleviated by inhibiting dopamine and/or norepinephrine and/or serotonin reuptake. Such disorders include, but are not limited to, addictive and substance abuse disorders such as nicotine addiction.

Additional disorders contemplated for treatment employing the methods of the invention are described, for example, in the Quick Reference to the Diagnostic Criteria from DSM-IV (Diagnostic and Statistical Manual of Mental Disorders, Fourth Edition), The American Psychiatric Association, Washington, D.C., 1994. Specific disorders whose definitions can be found in this reference are described below.

Addictive disorders amenable for treatment and/or prevention employing the methods and compositions of the invention include, but are not limited to, nicotine-related disorders.

Nicotine-related disorders include, but are not limited to, Nicotine Dependence, Nicotine Withdrawal, Nicotine Cessation, Nicotine Relapse, and Nicotine-Related Disorder not otherwise specified (NOS). The (+)-1-(3,4-dichlorophenyl)-3-azabicyclo[3.1.0]hexane agents described herein can be administered to help manage cravings associated with smoking reduction or cessation regimens, nicotine reduction or cessation regimens, nicotine withdrawal, or nicotine cravings, or consuming smoking cessation medicaments. The (+)-1-(3,4-dichlorophenyl)-3-azabicyclo[3.1.0]hexane agents of the present invention can likewise be used to alleviate symptoms of nicotine withdrawal. Such symptoms can include, but are not limited to, behavioral, emotional, cognitive, and physiological symptoms which emerge upon cessation or reduction of intake of nicotine. For example, such symptoms can include depression, irritability, anger, hostility, anxiety, nervousness, panic, poor concentration, disorientation, lightheadedness, sleep disturbances, constipation, mouth ulcers, dry mouth, sore throat-gums- or tongue, pain in limbs, sweating, depression, fatigue, fearfulness, sense of loss, craving tobacco, hunger, and coughing (body getting rid of the mucus clogging the lungs). While nicotine withdrawal symptoms can be relatively short lived, typically lasting from a few weeks to several months, nicotine cravings can endure long after nicotine cessation. Nicotine cravings can trigger urges that result in relapse.

With respect to nicotine-related disorders, including but not limited to, Nicotine Dependence, Nicotine Withdrawal, Nicotine Cessation, Nicotine Relapse, and Nicotine-Related Disorder not otherwise specified (NOS), (+)-1-(3,4-dichlorophenyl)-3-azabicyclo[3.1.0]hexane and pharmaceutically acceptable salts thereof can be used to decrease nicotine consumption and use of tobacco and other nicotine-containing products associated with such nicotine-related disorders. Accordingly, the present invention provides a method for treating or preventing nicotine consumption, comprising administering to a patient in need of such treatment or prevention an effective amount (+)-1-(3,4-dichlorophenyl)-3-azabicyclo[3.1.0]hexane or a pharmaceutically acceptable salt thereof. The present invention also provides a method for treating or preventing nicotine consumption and depression, comprising administering to a patient in need of such treatment or prevention an effective amount of (+)-1-(3,4-dichlorophenyl)-3-azabicyclo[3.1.0]hexane or a pharmaceutically acceptable salt thereof. The present invention further provides pharmaceutical compositions for treating or preventing nicotine consumption in a patient comprising an effective amount of (+)-1-(3,4-dichlorophenyl)-3-azabicyclo[3.1.0]hexane or a pharmaceutically acceptable salt thereof. The present invention also provides pharmaceutical compositions for treating or preventing nicotine consumption and depression in a patient comprising (+)-1-(3,4-dichlorophenyl)-3-azabicyclo[3.1.0]hexane or a pharmaceutically acceptable salt thereof.

In certain embodiments of the present invention, (+)-1-(3,4-dichlorophenyl)-3-azabicyclo[3.1.0]hexane or a pharmaceutically acceptable salt thereof can be used in combination therapy with at least one other therapeutic agent. (+)-1-(3,4-dichlorophenyl)-3-azabicyclo[3.1.0]hexane or a pharmaceutically acceptable salt thereof and the other therapeutic agent can act additively or, more preferably, synergistically. In a preferred embodiment, (+)-1-(3,4-dichlorophenyl)-3-azabicyclo[3.1.0]hexane or a pharmaceutically acceptable salt thereof is administered concurrently with the administration of another therapeutic agent, which can be part of the same composition as or in a different composition from that comprising (+)-1-(3,4-dichlorophenyl)-3-azabicyclo[3.1.0]hexane or a pharmaceutically acceptable salt thereof. The other therapeutic agent can be useful for treating and/or preventing (as defined herein) a secondary condition resulting from a disorder alleviated by inhibiting dopamine reuptake. In another embodiment, (+)-1-(3,4 dichlorophenyl)-3-azabicyclo[3.1.0]hexane or a pharmaceutically acceptable salt thereof is administered prior to or subsequent to administration of another therapeutic agent. As many of the disorders for which (+)-1-(3,4-dichlorophenyl)-3-azabicyclo[3.1.0]hexane and pharmaceutically acceptable salts thereof are useful in treating are chronic, in one embodiment combination therapy involves alternating between administering a composition comprising (+)-1-(3,4-dichlorophenyl)-3-azabicyclo[3.1.0]hexane or a pharmaceutically acceptable salt thereof and a composition comprising another therapeutic agent. The duration of administration of (+)-1-(3,4-dichlorophenyl)-3-azabicyclo[3.1.0]hexane, a pharmaceutically acceptable salt thereof, or the other therapeutic agent can be, e.g., one month, three months, six months, a year, or for more extended periods, such as the patient's lifetime. In certain embodiments, when (+)-1-(3,4-dichlorophenyl)-3-azabicyclo[3.1.0]hexane or a pharmaceutically acceptable salt thereof is administered concurrently with another therapeutic agent that potentially produces adverse side effects including, but not limited to, toxicity, the other therapeutic agent can advantageously be administered at a dose that falls below the threshold at which the adverse side effect is elicited.

The present invention also includes combinatorial formulations and coordinate administration methods which employ an effective amount of (+)-1-(3, 4-dichlorophenyl)-3-azabicyclo[3.1.0]hexane (or a pharmaceutically effective salt, solvate, hydrate, polymorph, or prodrug thereof), and one or more additional active agent(s) that is/are combinatorially formulated or coordinately administered with (+)-1-(3, 4-dichlorophenyl)-3-azabicyclo[3.1.0]hexane to yield a combinatorial formulation or coordinate administration method that is effective to prevent or treat nicotine consumption or both nicotine consumption and depression in a patient. Exemplary combinatorial formulations and coordinate treatment methods in this context include, for example, an effective amount of (+)-1-(3, 4-dichlorophenyl)-3-azabicyclo[3.1.0]hexane in combination with one or more additional or adjunctive treatment agents or methods for preventing or treating nicotine consumption or nicotine consumption, nicotine withdrawal symptoms, and depression in a patient, such as one or more anti-nicotine or anti-depressant agent(s) and/or therapeutic method(s).

In related embodiments of the invention, (+)-1-(3, 4-dichlorophenyl)-3-azabicyclo[3.1.0]hexane (or a pharmaceutically effective salt, solvate, hydrate, polymorph, or prodrug thereof) can be used in combination therapy with at least one other therapeutic agent or method. In this context, (+)-1-(3, 4-dichlorophenyl)-3-azabicyclo[3.1.0]hexane can be administered concurrently or sequentially with administration of a second therapeutic agent, for example a second agent that acts to treat or prevent nicotine consumption or both nicotine consumption and depression, or prevent or treat a different form of addiction or addictive behaviors, symptoms of addiction or addictive behaviors, or withdrawal from addictive substances or behaviors for which (+)-1-(3, 4-dichlorophenyl)-3-azabicyclo[3.1.0]hexane is administered. The (+)-1-(3, 4-dichlorophenyl)-3-azabicyclo[3.1.0]hexane and the second therapeutic agent can be combined in a single composition or administered in different compositions. The coordinate administration may be done simultaneously or sequentially in either order, and there may be a time period while only one or both (or all) active therapeutic agents, individually and/or collectively, exert their biological activities and therapeutic effects. A distinguishing aspect of all such coordinate treatment methods is that the (+)-1-(3, 4-dichlorophenyl)-3-azabicyclo[3.1.0]hexane exerts at least some detectable therapeutic activity towards treating, alleviating, decreasing, ameliorating, or preventing nicotine consumption, craving, withdrawal, both nicotine consumption and depression, or consumption, craving or withdrawal from another addiction or addictive behavior. Often targeted in these combinatorial treatment embodiments are comorbid psychiatric conditions, e.g., depression, or co-occurring addictive conditions including drug or behavioral addictions or compulsive disorders. Detectable therapeutic activity in this context may therefore be determined in conjunction with a secondary clinical response provided by the secondary therapeutic agent. Often, the coordinate administration of (+)-1-(3, 4-dichlorophenyl)-3-azabicyclo[3.1.0]hexane with a secondary therapeutic agent as contemplated herein will yield an enhanced therapeutic response beyond the therapeutic response elicited by either or both (+)-1-(3, 4-dichlorophenyl)-3-azabicyclo[3.1.0]hexane and/or secondary therapeutic agent alone.

Since (+)-1-(3, 4-dichlorophenyl)-3-azabicyclo[3.1.0]hexane may need to be administered to a patient in a long term treatment program for the purpose of preventing, alleviating, decreasing, ameliorating or treating nicotine consumption, craving withdrawal, both nicotine consumption and depression, or consumption, craving or withdrawal from another addiction or addictive behavior, in one embodiment combination therapy involves alternating between administering (+)-1-(3, 4-dichlorophenyl)-3-azabicyclo[3.1.0]hexane (or a pharmaceutically effective salt, solvate, hydrate, polymorph, or prodrug thereof) and a second therapeutic agent (i.e., alternating therapy regimens between the two drugs, e.g., at one week, one month, three month, six month, or one year intervals). Alternating drug regimens in this context will often reduce or even eliminate adverse side effects, such as toxicity, that may attend long-term administration of one or both drugs alone.

Useful secondary therapeutic agents for use as anti-nicotine agents include, but are not limited to, varenicline, bupropion, cytisine, anabasine, nortriptyline, mecamylamine, clonidine, anatabine with Yerba mate extract, compounds capable of affecting or selectively binding to a nicotinic acetylcholinergic receptor (nAChR) (see U.S. Patent Application Publication 20110274628), α1 receptor binding ligands, nicotine-alternative alkaloids, lobeline, compounds that selectively interact with neuronal nicotinic receptors (NNRs) such as nicotinic receptor desensitizing agents including, but not limited to, sazetidine A, and amantadine. Another useful anti-nicotine agent is a nicotinic immunogen and/or anti-nicotine antibodies. Anti-nicotine agents can also include nicotine replacement therapies, typically used in smoking-cessation, such as nicotine patches, gum, lozenges, nasal spray and inhaler.

Useful secondary therapeutic agents for use as anti-addictive-disorder agents in general (either against nicotine addiction and a co-morbid addiction, e.g., drug or behavioral, or selectively against a secondary addictive condition) further include, but are not limited to, tricyclic antidepressants; MAO inhibitors; glutamate agonists and antagonists, such as ketamine HCl, dextromethorphan, dextrorphan tartrate and dizocilpine (MK801); degrading enzymes, such as anesthetics and aspartate antagonists; GABA agonists, such as baclofen and muscimol HBr; reuptake blockers; degrading enzyme blockers; glutamate agonists, such as D-cycloserine, carboxyphenylglycine, L-glutamic acid, and cis-piperidinc-2,3-dicarboxylic acid; aspartate agonists; GABA antagonists such as gabazine (SR-95531), saclofen, bicuculline, picrotoxin, and (+) apomorphine HCl; α1 receptor binding agents such as 3-propoxy-β-carboline hydrochloride (3-PBC), muscarinic agents, and dopamine antagonists, such as spiperone HCl, haloperidol, and (−) sulpiride.

Other useful secondary therapeutic agents in this context include anti-depression agents such as, but not limited to, amitriptyline, isomers or racemic mixtures of citalopram, clomipramine, doxepine, duloxetine, imipramine, trimipramine, amoxapine, desipramine, maprotiline, nortriptyline, protripylinc, fluoxetine, fluvoxamine, mirtazepine, paroxetine, sertraline, venlafaxine, bupropion, nefazodone, trazodone, phenelzine, tranylcypromine, selegiline, clonidine, gabapentin, bicifadine and 2-pyridinyl[7-(pyridine-4-yl)pyrazolo[1,5-a]pyrimidin-3-yl]methanone compounds having at least one substituent on both the 2- and 4-pyridinyl rings. Useful classes of antidepressant agents include without limitation monoamine oxidase inhibitors, selective serotonin reuptake inhibitors, tricyclic antidepressants, tetracyclic antidepressants, norepinephrine uptake inhibitors, selective norepinephrine reuptake inhibitors, and serotonin and norepinephrine uptake inhibitors.

Other useful secondary therapeutic agents within the compositions and methods of the invention include anxiolytic agents such as, but not limited to, benzodiazepines, such as alprazolam, chlordiazepoxide, clonazepam, clorazepate, diazepam, halazepam, lorazepam, oxazepam, and prazepam; non-benzodiazepine agents, such as buspirone; and tranquilizers, such as barbiturates.

Other useful secondary therapeutic agents for use within the compositions and methods as described herein include anti-psychotic drugs such as, but not limited to, phenothiazines, such as chlorpromazine, mesoridazine besylate, thioridazine, acetophenazine maleate, fluphenazine, perphenazine, and trifluoperazine; thioxanthenes, such as chlorprothixene, and thiothixene; and other heterocyclic compounds, such as clozapine, haloperidol, loxapine, molindone, pimozide, and risperidone. Additional antipsychotic drugs include olanzapine, aripiprazole, quetiapine, and ziprasidone. Preferable anti-psychotic drugs include chlorpromazine HCl, thioridazine HCl, fluphenazine HCl, thiothixene HCl, and molindone HCl.

Administration of an effective amount of (+)-1-(3, 4-dichlorophenyl)-3-azabicyclo[3.1.0]hexane alone or in combination with a secondary therapeutic agent in accordance with the methods described herein to a mammalian subject presenting with one or more symptoms of a nicotine-related or other addictive disorder or CNS condition, will detectably decrease, eliminate, or prevent the targeted disorder(s) and/or associated symptom(s). In exemplary embodiments, administration of a (+)-1-(3, 4-dichlorophenyl)-3-azabicyclo[3.1.0]hexane composition to a suitable subject will yield a reduction in one or more target symptom(s) associated with a selected disorder, such as an addictive behavior or a craving for an addictive substance, by at least 10%, 20%, 30%, 50% or greater, up to a 75-90%, or 95% or greater, reduction in the targeted disorder or one or more target symptom(s), compared to placebo-treated or other suitable control subjects. In exemplary embodiments, this efficacy will be determined as reduced addictive behavior or activity, or alternatively as in the case of smoking cessation an improved cessation success rate of at least at least 10%, 20%, 30%, 50% or greater, up to a 75-90%, or 95% or greater compared to smoking cessation rates among placebo-treated control subjects. Comparable levels of efficacy using the methods and compositions described herein are contemplated for the treatment or prevention of the entire range of nicotine-related or addictive disorders and related conditions and symptoms as described above.

Administration of an effective amount of (+)-1-(3,4-dichlorophenyl)-3-azabicyclo[3.1.0]hexane azabicyclo[3.1.0]hexane or a pharmaceutically acceptable salt thereof, whether alone or in combination with a secondary therapeutic agent, to a patient will detectably treat or prevent, alleviate, decrease, or ameliorate nicotine consumption, craving, withdrawal, both nicotine consumption and depression, or consumption, craving or withdrawal from another addiction or addictive behavior in the patient. In exemplary embodiments, administration of a (+)-1-(3,4-dichlorophenyl)-3-azabicyclo[3.1.0]hexane azabicyclo[3.1.0]hexane or a pharmaceutically acceptable salt thereof, whether alone or in combination with a secondary therapeutic agent, to a patient will yield a reduction in nicotine consumption, craving for nicotine, both nicotine consumption and depression, or nicotine withdrawal symptoms by at least 0%, 20%, 30%, 50% or greater, up to a 75-90%, or 95% or greater, reduction in nicotine consumption, craving for nicotine, or both nicotine consumption and depression.

Cravings associated with addictive or compulsive behaviors (e.g., an urge or desire to smoke or to use drugs of abuse) can be assessed according to self-reported cravings, which provide a subjective index of a subject's motivational state. In some cases, such cravings can be assessed using one or more multidimensional scales such as the Questionnaire of Smoking Urges (QSU) developed by Tiffany & Drobes (Br. J. Addict. 86(11):1467-76 (1991)) which assesses the subject's desire to smoke and his or her expectancies of both positive and negative reinforcement from smoking and intention to smoke. Other questionnaires or indices useful for assessing cravings for nicotine or smoking can include the Measurement of Drug Craving scale (Sayette et al. 2000), Drug History Questionnaire (DHQ), Desires for Drug Questionnaire (Franken et al., Addict. Behav. 27:675-85 (2002)), Heaviness of Smoking Index (HSI), the Fagerstrom Test for Nicotine Dependence (FTND). In some cases, cravings or impulses associated with addictive and/or compulsive behaviors or behavioral modification protocols can be assessed using the Obsessive-Compulsive Beliefs Questionnaire-87 (OBQ-87).

Baseline nicotine craving assessment can be performed prior to administration of a composition provided herein, and additional craving assessments can be performed following administration of the composition. For example, a subject can be asked to report his or her “typical” craving experiences before, during, or after starting a behavioral modification program such as a smoking cessation or reduction program or a nicotine reduction or cessation program (either with or without using a smoking cessation medication (e.g., a transdermal nicotine patch), a chemical dependency program, or a program to reduce or eliminate other addictive and/or compulsive behavior. Baseline nicotine craving assessment is useful since individual subjects will differ in the way they use and respond to the questionnaire or other method of assessing the effects of a composition as described herein. Including a baseline score in the analysis can permit normalization of the relevant measures to each subject's standard. The accuracy of craving indices can be limited by the ability and willingness of a subject to accurately report his or her personal experience. Relapse to addictive behavior, frequency of use of nicotine or another addictive substance, the length of time since a subject last smoked or engaged in another addictive behavior, or the length of time since a subject last used nicotine or another addictive substance can also provide meaningful information for evaluating the effects of compositions described herein on weight management and appetite and craving control.

Individuals with addictive behaviors are at higher risk for depression and depressive symptoms due to withdrawal. (Glassman et al., 1990) Effectiveness of the methods and compositions described herein may further be demonstrated by a decrease in the effects of withdrawal including depression, anhedonia, anxiety and somatic symptoms associated with cessation of addictive behaviors (Stoker et al., 2008, West et al., 1984, Glassman, 1993, Glassman et al., 1990). Administration of an effective amount of (+)-1-(3,4-dichlorophenyl)-3-azabicyclo[3.1.0]hexane or a pharmaceutically acceptable salt thereof, whether alone or in combination with a secondary therapeutic agent, to a patient will detectably prevent or reduce withdrawal symptoms such as depression caused by ceasing an addictive behavior such as smoking by at least 0%, 20%, 30%, 50% or greater, up to a 75-90%, or 95% or greater, reduction in typical withdrawal symptoms including depression. A decrease in the effects of withdrawal, including depression, may be determined by conventional patient surveys or clinical scales to measure clinical indices of disorders in subjects. The methods and compositions of the invention will yield a reduction in one or more scores or selected values generated from such surveys or scales completed by subjects (indicating for example an incidence or severity of a selected disorder), by at least 10%, 20%, 30%, 50% or greater, up to a 75-90%, or 95% compared to correlative scores or values observed for control subjects treated with placebo or other suitable control treatment.

Useful patient surveys and clinical scales for comparative measurement of clinical indices of psychiatric disorders in subjects treated using the methods and compositions of the invention can include any of a variety of widely used and well known surveys and clinical scales. Among these useful tools are the Mini International Neuropsychiatric Interview© (MINI) (Sheehan et al., 1998); Clinical Global Impression scale (CGI) (Guy, W., ECDEU Assessment Manual for Psychopharmacology, DHEW Publication No. (ADM) 76-338, rev. 1976); Clinical Global Impression Severity of Illness (CGI-S) (Guy, 1976); Clinical Global Impression Improvement (CGI-I) (Guy, et al. 1976); Beck Depression Inventory (BDI) (Beck, 2006); Revised Hamilton Rating Scale for Depression (RHRSD) (Warren, 1994); Major Depressive Inventory (MDI) (Olsen et al. 2003); and Children's Depression Index (CDI) (Kovacs, et al. 1981); Hamilton Depression Rating Scale© (HDRS) (Hamilton, M., J. Neurol. Neurosurg. Psychiatr. 23:56-62, 1960; Hamilton, M., Br. J. Soc. Clin. Psychol. 6:278-296, 1967); Montgomery-Asberg Depression Rating Scale© (MADRS) (Montgomery and Asberg, 1979); Beck Scale for Suicide Ideation® (BSS) (Beck and Steer, 1991 Columbia-Suicide Severity Rating Scale© (C-SSRS) or Columbia Classification Algorithm of Suicide Assessment© (C CASA) (Posner, K, et al., 2007); Sheehan-Suicidality Tracking Scale© (S-SST) (Coric et al., 2009); Beck Hopelessness Scale© (BHS) (Beck, Steer, 1988); Geriatric Depression Scale (GDS) (Yesavage, J. A. et al., J. Psychiatr. Res. 17:37-49, 1983); and the HAM-D scale for depression (Hamilton, 1960).

The methods and compositions of the invention will yield a reduction in one or more scores or values generated from these clinical surveys (using any single scale or survey, or any combination of one or more of the surveys described above) by at least 10%, 20%, 30%, 50% or greater, up to a 75-90%, or 95% compared to correlative scores or values observed for control subjects treated with placebo or other suitable control treatment. In prophylactic treatment, the methods and compositions of the invention will yield a stabilization or diminished change in the scores or values generated from these clinical surveys.

Additionally, effectiveness of the compositions and methods described herein using amitifadine ((+)-1-(3, 4-dichlorophenyl)-3-azabicyclo[3.1.0]hexane) may be determined through the use of animal models. Given the prevalence of depression among those with addictive behaviors or during withdrawal from addictive behaviors, animal models such as the Forced Swim Test or other animal models of depression are useful in demonstrating the effectiveness of amitifadine ((+)-1-(3, 4-dichlorophenyl)-3-azabicyclo[3.1.0]hexane) in the treatment of the effects of withdrawal from addictive behaviors. As shown in Example I, below, some rats (P) were sensitized to nicotine while others (NP) were not. Example VII provides evidence from the FST in P rats that (+)-1-(3, 4-dichlorophenyl)-3-azabicyclo[3.1.0]hexane attenuates the immobility that occurs upon administration of nicotine in the rats. These results demonstrate that (+)-1-(3, 4-dichlorophenyl)-3-azabicyclo[3.1.0]hexane would be efficacious in reducing depressive symptoms in humans that typically accompany nicotine withdrawal.

The effect of amitifadine ((+)-1-(3, 4-dichlorophenyl)-3-azabicyclo[3.1.0]hexane) may additionally be measured using models such as an intracranial self-stimulation (ICSS) model which is used to understand how pharmacological or molecular manipulations affect the function of the brain reward systems or behavioral sensitization models which create behavioral effects that are indicative of chronic drug use. (Schroeder et al., 2001; Miller et al., 2001). Behavioral sensitization models have been used to show that chronic exposure to drugs of abuse such as nicotine in animal models can have long-lasting effects on the neuromechanisms of reward pathways similar to the effects of nicotine addiction and is thus a useful animal model for determining the effectiveness of compositions in desensitizing an animal to a particular addiction or changing the behavior patterns regarding a particular addictive behavior and/or substance.

In the behavioral sensitization model as shown in Example II, below, (+)-1-(3, 4-dichlorophenyl)-3-azabicyclo[3.1.0]hexane reduced nicotine sensitization in P rats. The ability of (+)-1-(3,4-dichlorophenyl)-3-azabicyclo[3.1.0]hexane to attenuate nicotine induced sensitization in the well-accepted P rat model indicates that (+)-1-(3,4-dichlorophenyl)-3-azabicyclo[3.1.0]hexane can block the neurochemical effects of nicotine. Accordingly, (+)-1-(3,4-dichlorophenyl)-3-azabicyclo[3.1.0]hexane may be efficacious in treating nicotine-related disorders in humans.

Intracranial self-stimulation and behavioral sensitization were combined to evaluate the potentiating effects of nicotine as a drug of abuse. As shown in Example III, below, P rats have an increased response in the intracranial self-stimulation (ICSS) paradigm compared to NP rats. Examples IV and VI, below, show that nicotine potentiation of ICSS in P rats was reduced by (+)-1-(3, 4-dichlorophenyl)-3-azabicyclo[3.1.0]hexane, and that the compound attenuated nicotine-induced abstinence effects in ICSS in P rats. These data demonstrate the utility of (+)-1-(3, 4-dichlorophenyl)-3-azabicyclo[3.1.0]hexane for antagonizing nicotine's actions and reducing abstinence effects that occur upon withdrawal of nicotine consumption.

The effectiveness of the compositions and methods as described herein may further be demonstrated through the use of animal behavioral paradigms. Animal behavioral paradigms are used to explore the positive and negative reinforcing actions of drugs. Using a self-administration paradigm, rats were trained that a correct lever press resulted in the delivery of a nicotine infusion (0.03 mg/kg/infusion) on a fixed ratio (FR) 1 schedule of reinforcement and the activation of a feedback tone for 0.05 s. As shown in Example IX, amitifadine ((+)-1-(3, 4-dichlorophenyl)-3-azabicyclo[3.1.0]hexane) significantly reduced nicotine self-administration in Sprague-Dawley rats at doses that were not seen to cause locomotor hypoactivity or decreased response to food. This is despite the fact that amitifadine appears to have no direct effect on nicotinic receptors (Example VIII). While not wishing to be bound, it is theorized that the inhibition of reuptake of the monoaminergic neurotransmitters was important for the reduction of nicotine self-administration.

(+)-1-(3, 4-dichlorophenyl)-3-azabicyclo[3.1.0]hexane agents for the treatment of addiction and/or the symptoms of withdrawal may be administered by any means generally used. Suitable routes of administration for a (+)-1-(3, 4-dichlorophenyl)-3-azabicyclo[3.1.0]hexane agent in the methods disclosed herein include, but are not limited to, oral, buccal, nasal, aerosol, topical, transdermal, mucosal, injectable, slow release, controlled release, iontophoresis, sonophoresis, and other conventional delivery routes, devices and methods. Injectable delivery methods are also contemplated, including but not limited to, intravenous, intramuscular, intraperitoneal, intraspinal, intrathecal, intracerebroventricular, intraarterial, and subcutaneous injection.

Suitable effective unit dosage amounts of a (+)-1-(3, 4-dichlorophenyl)-3-azabicyclo[3.1.0]hexane compound used as disclosed herein for mammalian subjects may range from about 1 to about 1800 mg, about 10 to about 1800 mg, 25 to about 1800 mg, about 50 to about 1000 mg, about 75 to about 900 mg, about 100 to about 750 mg, or about 150 to about 500 mg. In certain embodiments, the effective dosage will be selected within narrower ranges of, for example, about 5 to about 10 mg, 10 to about 25 mg, about 30 to about 50 mg, about 10 to about 300 mg, about 25 to about 300 mg, about 50 to about 100 mg, about 100 to about 250 mg, or about 250 to about 500 mg. These and other effective unit dosage amounts may be administered in a single dose, or in the form of multiple daily, weekly or monthly doses, for example in a dosing regimen comprising from 1 to 4, or 2-3, doses administered per day, per week, or per month. In exemplary embodiments, dosages of about 10 to about 25 mg, about 30 to about 50 mg, about 25 to about 150, about 50 to about 100 mg, about 100 to about 250 mg, or about 250 to about 500 mg, are administered one, two, three, or four times per day. In more detailed embodiments, dosages of about 50-75 mg, about 100-200 mg, about 250-400 mg, or about 400-600 mg are administered once or twice daily. In further detailed embodiments, dosages of about 50-100 mg are administered twice daily. In alternate embodiments, dosages are calculated based on body weight, and may be administered, for example, in amounts from about 0.5 mg/kg to about 20 mg/kg per day, 1 mg/kg to about 15 mg/kg per day, 1 mg/kg to about 10 mg/kg per day, 2 mg/kg to about 20 mg/kg per day, 2 mg/kg to about 10 mg/kg per day or 3 mg/kg to about 15 mg/kg per day.

The amount, timing, and mode of delivery of compositions comprising an effective amount of a (+)-1-(3, 4-dichlorophenyl)-3-azabicyclo[3.1.0]hexane agent as used in the methods described herein will be routinely adjusted on an individual basis, depending on such factors as weight, age, gender, and condition of the individual, the acuteness of the condition to be treated and/or related symptoms, whether the administration is prophylactic or therapeutic, and on the basis of other factors known to effect drug delivery, absorption, pharmacokinetics, including half-life, and efficacy. An effective dose or multi-dose treatment regimen for the compounds of the invention will ordinarily be selected to approximate a minimal dosing regimen that is necessary and sufficient to substantially prevent or alleviate one or more symptom(s) of a neurological or psychiatric condition in the subject, as described herein. Thus, following administration of a (+)-1-(3, 4-dichlorophenyl)-3-azabicyclo[3.1.0]hexane compound or pharmaceutically acceptable salt thereof according to the formulations and methods herein, test subjects will exhibit a 10%, 20%, 30%, 50% or greater reduction, up to a 75-90%, or 95% or greater, reduction, in one or more symptoms associated with a targeted monoamine neurotransmitter influenced disorder or other neurological or psychiatric condition, compared to placebo-treated or other suitable control subjects.

Pharmaceutical dosage forms of a compound used in the present invention may optionally include excipients recognized in the art of pharmaceutical compounding as being suitable for the preparation of dosage units as discussed above. Such excipients include, without intended limitation, binders, fillers, lubricants, emulsifiers, suspending agents, sweeteners, flavorings, preservatives, buffers, wetting agents, disintegrants, effervescent agents and other conventional excipients and additives.

Pharmaceutical dosage forms of a (+)-1-(3, 4-dichlorophenyl)-3-azabicyclo[3.1.0]hexane composition may include inorganic and organic acid addition salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; organic acid salts such as acetate, citrate, lactate, succinate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate, tartrate, gluconate and the like.

Within various combinatorial or coordinate treatment methods disclosed herein, the additional therapeutic agent and a (+)-1-(3, 4-dichlorophenyl)-3-azabicyclo[3.1.0]hexane compound or a pharmaceutically acceptable salt thereof may each be administered by any of a variety of delivery routes and modes, which may be the same or different for each agent.

An additional psychotherapeutic compound and/or a (+)-1-(3, 4-dichlorophenyl)-3-azabicyclo[3.1.0]hexane administered according to the present invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets and capsules, which may be prepared by any conventional method of preparing pharmaceutical oral unit dosage forms can be utilized in preparing oral unit dosage forms. Oral unit dosage forms, such as tablets or capsules, may contain one or more conventional additional formulation ingredients, including, but are not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and preferably in less than thirty seconds. By effervescent agent is meant a couple, typically an organic acid and a carbonate or bicarbonate.

A (+)-1-(3, 4-dichlorophenyl)-3-azabicyclo[3.1.0]hexane composition as disclosed herein can be prepared and administered in any of a variety of inhalation or nasal delivery forms known in the art. Devices capable of depositing aerosolized formulations of a triple reuptake inhibitor compound or a pharmaceutically acceptable salt thereof of the invention in the sinus cavity or pulmonary alveoli of a patient include metered dose inhalers, nebulizers, dry powder generators, sprayers, and the like. Pulmonary delivery to the lungs for rapid transit across the alveolar epithelium into the blood stream may be particularly useful in treating impending episodes of depression. Methods and compositions suitable for pulmonary delivery of drugs for systemic effect are well known in the art. Suitable formulations, wherein the carrier is a liquid, for administration, as for example, a nasal spray or as nasal drops, may include aqueous or oily solutions of a compound of the present invention, and any additional active or inactive ingredient(s).

Intranasal delivery permits the passage of active compounds as disclosed herein into the blood stream directly after administering an effective amount of the compound to the nose, without requiring the product to be deposited in the lung. In addition, intranasal delivery can achieve direct, or enhanced, delivery of the active compound to the central nervous system. In these and other embodiments, intranasal administration of the compounds of the invention may be advantageous for treating disorders influenced by monoamine neurotransmitters, by providing for rapid absorption and delivery.

For intranasal and pulmonary administration, a liquid aerosol formulation will often contain an active compound as described herein combined with a dispersing agent and/or a physiologically acceptable diluent. Alternative, dry powder aerosol formulations may contain a finely divided solid form of the subject compound and a dispersing agent allowing for the ready dispersal of the dry powder particles. With either liquid or dry powder aerosol formulations, the formulation must be aerosolized into small, liquid or solid particles in order to ensure that the aerosolized dose reaches the mucous membranes of the nasal passages or the lung. The term “aerosol particle” is used herein to describe a liquid or solid particle suitable of a sufficiently small particle diameter, e.g., in a range of from about 2-5 microns, for nasal or pulmonary distribution to targeted mucous or alveolar membranes. Other considerations include the construction of the delivery device, additional components in the formulation, and particle characteristics. These aspects of nasal or pulmonary administration of drugs are well known in the art, and manipulation of formulations, aerosolization means, and construction of delivery devices, is within the level of ordinary skill in the art.

Yet additional methods of the invention are provided for topical administration of a (+)-1-(3, 4-dichlorophenyl)-3-azabicyclo[3.1.0]hexane compound or a pharmaceutically acceptable salt thereof. Topical compositions may comprise a compound as described herein and any other active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise a compound as disclosed herein dissolved or dispersed in water or other solvent or liquid to be incorporated in the topical composition or delivery device. It can be readily appreciated that the transdermal route of administration may be enhanced by the use of a dermal penetration enhancer known to those skilled in the art. Formulations suitable for such dosage forms incorporate excipients commonly utilized therein, particularly means, e.g. structure or matrix, for sustaining the absorption of the drug over an extended period of time, for example 24 hours.

Yet additional formulations of a compound used in the present invention are provided for parenteral administration, including aqueous and non-aqueous sterile injection solutions which may optionally contain anti-oxidants, buffers, bacteriostats and/or solutes which render the formulation isotonic with the blood of the mammalian subject; aqueous and non-aqueous sterile suspensions which may include suspending agents and/or thickening agents; dispersions; and emulsions. The formulations may be presented in unit-dose or multi-dose containers. Pharmaceutically acceptable formulations and ingredients will typically be sterile or readily sterilizable, biologically inert, and easily administered. Parenteral preparations typically contain buffering agents and preservatives, and may be lyophilized for reconstitution at the time of administration.

Parental formulations may also include polymers for extended release following parenteral administration. Such polymeric materials are well known to those of ordinary skill in the pharmaceutical compounding arts. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

Within exemplary compositions and dosage forms used in the methods of the invention, a (+)-1-(3, 4-dichlorophenyl)-3-azabicyclo[3.1.0]hexane compound or a pharmaceutically acceptable salt thereof for treating disorders disclosed herein is administered in an extended release or sustained release formulation. In these formulations, the sustained release composition of the formulation provides therapeutically effective plasma levels of the active compound or a pharmaceutically acceptable salt thereof over a sustained delivery period of approximately 8 hours or longer, or over a sustained delivery period of approximately 18 hours or longer, up to a sustained delivery period of approximately 24 hours or longer, to enhance efficacy of the subject compositions and methods for abating addictive behaviors or relapse (e.g., smoking), cravings and other withdrawal symptoms.

In exemplary embodiments, a (+)-1-(3, 4-dichlorophenyl)-3-azabicyclo[3.1.0]hexane compound or a pharmaceutically acceptable salt thereof is combined with a sustained release vehicle, matrix, binder, or coating material. As used herein, the term “sustained release vehicle, matrix, binder, or coating material” refers to any vehicle, matrix, binder, or coating material that effectively, significantly delays dissolution of the active compound in vitro, and/or delays, modifies, or extends delivery of the active compound into the blood stream (or other in vivo target site of activity) of a subject following administration (e.g., oral administration), in comparison to dissolution and/or delivery provided by an “immediate release” formulation, as described herein, of the same dosage amount of the active compound. Accordingly, the term “sustained release vehicle, matrix, binder, or coating material” as used herein is intended to include all such vehicles, matrices, binders and coating materials known in the art as “sustained release”, “delayed release”, “slow release”, “extended release”, “controlled release”, “modified release”, and “pulsatile release” vehicles, matrices, binders and coatings.

In one aspect, the current invention comprises methods using an oral sustained release dosage composition for administering a (+)-1-(3, 4-dichlorophenyl)-3-azabicyclo[3.1.0]hexane compound or a pharmaceutically acceptable salt thereof. In a related aspect, the invention comprises a method of reducing one or more side effects that attend administration of an oral dosage form of a (+)-1-(3, 4-dichlorophenyl)-3-azabicyclo[3.1.0]hexane compound or a pharmaceutically acceptable salt thereof by employing a sustained release formulation. Within this method, following oral administration of a (+)-1-(3, 4-dichlorophenyl)-3-azabicyclo[3.1.0]hexane compound or a pharmaceutically acceptable salt thereof, the active agent is released in a sustained, delayed, gradual or modified release delivery mode into the gastrointestinal tract (e.g., the intestinal lumen) of the subject over a period of hours, during which the active compound is sustained at a therapeutic concentration in a blood plasma, tissue, organ or other target site of activity (e.g., a central nervous system tissue, fluid or compartment) in the patient. When following this method, the side effect profile of the (+)-1-(3, 4-dichlorophenyl)-3-azabicyclo[3.1.0]hexane is less than a side effect profile of an equivalent dose of the compound in an immediate release oral dosage form.

In certain embodiments, a (+)-1-(3, 4-dichlorophenyl)-3-azabicyclo[3.1.0]hexane compound or a pharmaceutically acceptable salt thereof is released from the sustained release compositions and dosage forms of the invention and delivered into the blood plasma or other target site of activity in the subject at a sustained therapeutic level over a period of at least about 6 hours, often over a period of at least about 8 hours, at least about 12 hours, or at least about 18 hours, and in other embodiments over a period of about 24 hours or greater. By sustained therapeutic level is meant a plasma concentration level of at least a lower end of a therapeutic dosage range as exemplified herein. In alternate embodiments of the invention, the sustained release compositions and dosage forms will yield a therapeutic level of a (+)-1-(3, 4-dichlorophenyl)-3-azabicyclo[3.1.0]hexane compound or a pharmaceutically acceptable salt thereof following administration to a mammalian subject in a desired dosage amount (e.g., 5, 10, 25, 50, 100, 200, 400, 600, or 800 mg) that yields a minimum plasma concentration that is known to be associated with clinical efficacy, e.g. for treating nicotine addiction, over a period of at least about 6 hours, at least about 8 hours, at least about 12 hours, at least about 18 hours, or up to 24 hours or longer.

In certain embodiments, a (+)-1-(3, 4-dichlorophenyl)-3-azabicyclo[3.1.0]hexane compound or a pharmaceutically acceptable salt thereof is released from the compositions and dosage forms disclosed herein and delivered into the blood plasma or other target site of activity in the subject (including, but not limited to, areas of the brain such as the prefrontal cortex, frontal cortex, thalamus, striatum, ventral tegmental area, other cortical areas, hippocampus, hypothalamus, or nucleus accumbens) in a sustained release profile characterized in that from about 0% to 20% of the active compound is released and delivered (as determined, e.g., by measuring blood plasma levels) within in 0 to 2 hours, from 20% to 50% of the active compound is released and delivered within about 2 to 12 hours, from 50% to 85% of the active compound is released and delivered within about 3 to 20 hours, and greater than 75% of the active compound is released and delivered within about 5 to 18 hours.

In more detailed embodiments of the invention, compositions and oral dosage forms of a (+)-1-(3, 4-dichlorophenyl)-3-azabicyclo[3.1.0]hexane compound or a pharmaceutically acceptable salt thereof are provided, wherein the compositions and dosage forms, after ingestion, provide a curve of concentration of a (+)-1-(3, 4-dichlorophenyl)-3-azabicyclo[3.1.0]hexane compound or a pharmaceutically acceptable salt thereof agents over time, the curve having an area under the curve (AUC) which is approximately proportional to the dose of the (+)-1-(3, 4-dichlorophenyl)-3-azabicyclo[3.1.0]hexane compound or a pharmaceutically acceptable salt thereof administered, and a maximum concentration (Cₘₐₓ) that is proportional to the dose of the (+)-1-(3, 4-dichlorophenyl)-3-azabicyclo[3.1.0]hexane compound or a pharmaceutically acceptable salt thereof administered.

In other detailed embodiments, the Cₘₐₓ of a (+)-1-(3, 4-dichlorophenyl)-3-azabicyclo[3.1.0]hexane compound or a pharmaceutically acceptable salt thereof provided after oral delivery of a composition or dosage form of the invention is less than about 80%, often less than about 75%, in some embodiments less than about 60%, or 50%, of a Cₘₐₓ obtained after administering an equivalent dose of the active compound in an immediate release oral dosage form.

Within exemplary embodiments of the invention, the compositions and dosage forms containing of a (+)-1-(3, 4-dichlorophenyl)-3-azabicyclo[3.1.0]hexane compound or a pharmaceutically acceptable salt thereof and a sustained release vehicle, matrix, binder, or coating will yield sustained delivery of the active compound such that, following administration of the composition or dosage form to a mammalian treatment subject, the Cₘₐₓ of the (+)-1-(3, 4-dichlorophenyl)-3-azabicyclo[3.1.0]hexane compound or a pharmaceutically acceptable salt thereof in the treatment subject is less than about 80% of a Cₘₐₓ provided in a control subject after administration of the same amount of the (+)-1-(3, 4-dichlorophenyl)-3-azabicyclo[3.1.0]hexane compound or a pharmaceutically acceptable salt thereof in an immediate release formulation.

As used herein, the term “immediate release dosage form” refers to a dosage form of a (+)-1-(3, 4-dichlorophenyl)-3-azabicyclo[3.1.0]hexane compound or a pharmaceutically acceptable salt thereof wherein the active compound readily dissolves upon contact with a liquid physiological medium, for example phosphate buffered saline (PBS) or natural or artificial gastric fluid. In certain embodiments, an immediate release formulation will be characterized in that at least 70% of the active compound will be dissolved within a half hour after the dosage form is contacted with a liquid physiological medium. In alternate embodiments, at least 80%, 85%, 90% or more, or up to 100%, of the active compound in an immediate release dosage form will dissolve within a half hour following contact of the dosage form with a liquid physiological medium in an art-accepted in vitro dissolution assay. These general characteristics of an immediate release dosage form will often relate to powdered or granulated compositions of a (+)-1-(3, 4-dichlorophenyl)-3-azabicyclo[3.1.0]hexane compound or a pharmaceutically acceptable salt thereof in a capsulated dosage form, for example in a gelatin-encapsulated dosage form, where dissolution will often be relatively immediate after dissolution/failure of the gelatin capsule. In alternate embodiments, the immediate release dosage form may be provided in the form of a compressed tablet, granular preparation, powder, or even liquid dosage form, in which cases the dissolution profile will often be even more immediate (e.g., wherein at least 85%-95% of the active compound is dissolved within a half hour).

In additional embodiments of the invention, an immediate release dosage form will include compositions wherein the (+)-1-(3, 4-dichlorophenyl)-3-azabicyclo[3.1.0]hexane compound or a pharmaceutically acceptable salt thereof is not admixed, bound, coated or otherwise associated with a formulation component that substantially impedes in vitro or in vivo dissolution and/or in vivo bioavailability of the active compound. Within certain embodiments, a (+)-1-(3, 4-dichlorophenyl)-3-azabicyclo[3.1.0]hexane compound or a pharmaceutically acceptable salt thereof will be provided in an immediate release dosage form that does not contain significant amounts of a sustained release vehicle, matrix, binder or coating material. In this context, the term “significant amounts of a sustained release vehicle, matrix, binder or coating material” is not intended to exclude any amount of such materials, but an amount sufficient to impede in vitro or in vivo dissolution of a (+)-1-(3, 4-dichlorophenyl)-3-azabicyclo[3.1.0]hexane compound or a pharmaceutically acceptable salt thereof in a formulation containing such materials by at least 5%, often at least 10%, and up to at least 15%-20% compared to dissolution of the (+)-1-(3, 4-dichlorophenyl)-3-azabicyclo[3.1.0]hexane compound or a pharmaceutically acceptable salt thereof when provided in a composition that is essentially free of such materials.

In alternate embodiments of the invention, an immediate release dosage form of a (+)-1-(3, 4-dichlorophenyl)-3-azabicyclo[3.1.0]hexane compound or a pharmaceutically acceptable salt thereof may be any dosage form comprising the active compound which fits the FDA Biopharmaceutics Classification System (BCS) Guidance definition (see, e.g., http://www.fda.gov/cder/OPS/BCS_guidance.htm) of a “high solubility substance in a rapidly dissolving formulation.” In exemplary embodiments, an immediate release formulation of a (+)-1-(3, 4-dichlorophenyl)-3-azabicyclo[3.1.0]hexane compound or a pharmaceutically acceptable salt thereof according to this aspect of the invention will exhibit rapid dissolution characteristics according to BCS Guidance parameters, such that at least approximately 85% of (+)-1-(3, 4-dichlorophenyl)-3-azabicyclo[3.1.0]hexane compound or a pharmaceutically acceptable salt thereof in the formulation will go into a test solution within about 30 minutes at pH 1, pH 4.5, and pH 6.8.

The sustained release dosage forms used in the methods of the invention can take any form as long as one or more of the dissolution, release, delivery and/or pharmacokinetic property(ies) identified above are satisfied. Within illustrative embodiments, the composition or dosage form can comprise a (+)-1-(3, 4-dichlorophenyl)-3-azabicyclo[3.1.0]hexane compound or a pharmaceutically acceptable salt thereof combined with any one or combination of: a drug-releasing polymer, matrix, bead, microcapsule, or other solid drug-releasing vehicle; drug-releasing tiny timed-release pills or mini-tablets; compressed solid drug delivery vehicle; controlled release binder; multi-layer tablet or other multi-layer or multi-component dosage form; drug-releasing lipid; drug-releasing wax; and a variety of other sustained drug release materials as contemplated herein, or formulated in an osmotic dosage form.

The present invention thus encompasses a broad range of sustained release compositions and dosage forms a (+)-1-(3, 4-dichlorophenyl)-3-azabicyclo[3.1.0]hexane compound or a pharmaceutically acceptable salt thereof, which in certain embodiments are adapted for providing sustained release of the active compound(s) following, e.g., oral administration. Sustained release vehicles, matrices, binders and coatings for use in accordance with the invention include any biocompatible sustained release material which is inert to the active agent and which is capable of being physically combined, admixed, or incorporated with the active compound. Useful sustained release materials may be dissolved, degraded, disintegrated, and/or metabolized slowly under physiological conditions following delivery (e.g., into a gastrointestinal tract of a subject, or following contact with gastric fluids or other bodily fluids). Useful sustained release materials are typically non-toxic and inert when contacted with fluids and tissues of mammalian subjects, and do not trigger significant adverse side effects such as irritation, immune response, inflammation, or the like. They are typically metabolized into metabolic products which are biocompatible and easily eliminated from the body.

In certain embodiments, sustained release polymeric materials are employed as the sustained release vehicle, matrix, binder, or coating (see, e.g., “Medical Applications of Controlled Release,” Langer and Wise (eds.), CRC Press., Boca Raton, Fla. (1974); “Controlled Drug Bioavailability,” Drug Product Design and Performance, Smolen and Ball (eds.), Wiley, N.Y. (1984); Ranger and Peppas, 1983, J Macromol. Sci. Rev. Macromol Chem. 23:61; see also Levy et al., 1985, Science 228: 190; During et al., 1989, Ann. Neurol. 25:351; Howard et al, 1989, J. Neurosurg. 71:105). Within exemplary embodiments, useful polymers for co-formulating with a (+)-1-(3, 4-dichlorophenyl)-3-azabicyclo[3.1.0]hexane compound or a pharmaceutically acceptable salt thereof to yield a sustained release composition or dosage form include, but are not limited to, ethylcellulose, hydroxyethyl cellulose; hydroxyethylmethyl cellulose; hydroxypropyl cellulose; hydroxypropylmethyl cellulose; hydroxypropylmethyl cellulose phthalate; hydroxypropylmethylcellulose acetate succinate; hydroxypropylmethylcellulose acetate phthalate; sodium carboxymethylcellulose; cellulose acetate phthalate; cellulose acetate trimellitate; polyoxyethylene stearates; polyvinyl pyrrolidone; polyvinyl alcohol; copolymers of polyvinyl pyrrolidone and polyvinyl alcohol; polymethacrylate copolymers; and mixtures thereof.

In other embodiments of the invention, (+)-1-(3, 4-dichlorophenyl)-3-azabicyclo[3.1.0]hexane may be encapsulated for delivery in microcapsules, microparticles, or microspheres, prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macroemulsions.

In yet additional embodiments of the invention, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release). Enteric coatings may function as a means for mediating sustained release of a (+)-1-(3, 4-dichlorophenyl)-3-azabicyclo[3.1.0]hexane compound or a pharmaceutically acceptable salt thereof by providing one or more barrier layers, which may be located entirely surrounding the active compound, between layers of a multi-layer solid dosage form (see below), and/or on one or more outer surfaces of one or multiple layers of a multi-layer solid dosage form (e.g., on end faces of layers of a substantially cylindrical tablet). Such barrier layers may, for example, be composed of polymers which are either substantially or completely impermeable to water or aqueous media, or are slowly erodible in water or aqueous media or biological liquids and/or which swell in contact with water or aqueous media. Suitable polymers for use as a barrier layer include acrylates, methacrylates, copolymers of acrylic acid, celluloses and derivatives thereof such as ethylcelluloses, cellulose acetate propionate, polyethylenes and polyvinyl alcohols etc. Additional enteric coating materials for mediating sustained release of a (+)-1-(3, 4-dichlorophenyl)-3-azabicyclo[3.1.0]hexane or a pharmaceutically acceptable salt thereof include coatings in the form of polymeric membranes, which may be semipermeable, porous, or asymmetric membranes (see, e.g., U.S. Pat. No. 6,706,283), and other polymeric coating materials and devices made from, for example, polyethylene glycol, polypropylene glycol, copolymers of polyethylene glycol and polypropylene glycol, poly(vinylpyrrolidone), ethyl cellulose, hydroxyethyl cellulose, hydroxypropyl cellulose, carboxymethyl cellulose, carboxymethylethyl cellulose, starch, dextran, dextrin, chitosan, collagen, gelatin, bromelain, cellulose acetate, unplasticized cellulose acetate, plasticized cellulose acetate, reinforced cellulose acetate, cellulose acetate phthalate, cellulose acetate trimellitate, hydroxypropylmethylcellulose, hydroxypropylmethyl-cellulose phthalate, hydroxypropylmethylcellulose acetate succinate, hydroxypropylmethylcellulose acetate trimellitate, cellulose nitrate, cellulose diacetate, cellulose triacetate, agar acetate, amylose triacetate, beta glucan acetate, beta glucan triacetate, acetaldehyde dimethyl acetate, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate chloroacetate, cellulose acetate ethyl oxalate, cellulose acetate methyl sulfonate, cellulose acetate butyl sulfonate, cellulose acetate propionate, cellulose acetate p-toluene sulfonate, triacetate of locust gum bean, cellulose acetate with acetylated hydroxyethyl cellulose, hydroxlated ethylene-vinylacetate, cellulose acetate butyrate, polyalkenes, polyethers, polysulfones, polyethersulfones, polystyrenes, polyvinyl halides, polyvinyl esters and ethers, natural waxes and synthetic waxes.

In a particular embodiment described below in Example XII, a formulation is provided for an oral unit dosage extended release tablet of an HCl salt of (+)-1-(3,4-dichlorophenyl)-3-azabicyclo[3.1.0]hexane. In this formulation hydroxypropylmethyl cellulose is used as an illustrative sustained release vehicle, while microcrystalline cellulose and starch is used as exemplary carrier/excipient agents. In this exemplary formulation a 350 mg tablet is provided that contains 100 mg of (+)-1-(3,4-dichlorophenyl)-3-azabicyclo[3.1.0]hexane (HCl salt), 105 mg of Methocel Premium CR K4 or K100, 71.5 mg Microcrystalline Cellulose, 70 mg pregelatinized starch 1500, 1.75 mg colloidal silicon dioxide, 1.75 mg magnesium stearate, and an optional coating, such as Opadry II White. Thus, the formulation uses 30% hydroxypropylmethyl cellulose (% of total weight of the tablet ingredients). According to this exemplary embodiment, an oral extended release tablet of (+)-1-(3,4-dichlorophenyl)-3-azabicyclo[3.1.0]hexane HCl (or other pharmaceutically acceptable salt) will include an amount of about 15-45%, 25-35%, or 30% of hydroxypropyl methyl cellulose of total weight of the tablet ingredients. An oral extended release tablet of (+)-1-(3,4-dichlorophenyl)-3-azabicyclo[3.1.0]hexane HCl or other pharmaceutically acceptable salt will further contain about 25 to 200 mg, 50 to 150 mg, or 100 mg of an active ingredient of (+)-1-(3,4-dichlorophenyl)-3-azabicyclo[3.1.0]hexane HCl or other pharmaceutically acceptable salt. An oral extended release tablet of (+)-1-(3,4-dichlorophenyl)-3-azabicyclo[3.1.0]hexane HCl or other pharmaceutically acceptable salt will additionally contain from about 30-50% or 40% of pharmaceutically acceptable carrier. An extended release profile of the formulation of Example XII is demonstrated by dissolution studies shown in Example XIII. Those studies demonstrate that the formulation of Example XII does indeed achieve an extended release commensurate with a tablet to be administered once per day.

The pharmaceutical compositions and dosage forms used in the current invention will typically be provided for administration in a sterile or readily sterilizable, biologically inert, and easily administered form.

In other embodiments the invention provides pharmaceutical kits for reducing symptoms in a human subject suffering from a disorder affected by monoamine neurotransmitters, including depression. The kits comprise a (+)-1-(3, 4-dichlorophenyl)-3-azabicyclo[3.1.0]hexane compound or a pharmaceutically acceptable salt thereof in an effective amount, and a container means for containing the (+)-1-(3, 4-dichlorophenyl)-3-azabicyclo[3.1.0]hexane compound or a pharmaceutically acceptable salt thereof for coordinate administration to the said subject (for example a container, divided bottle, or divided foil pack). The container means can include a package bearing a label or insert that provides instructions for multiple uses of the kit contents to treat the disorder and reduce symptoms in the subject. In more detailed embodiments, the (+)-1-(3, 4-dichlorophenyl)-3-azabicyclo[3.1.0]hexane compound or a pharmaceutically acceptable salt thereof is admixed or co-formulated in a single, combined dosage form, for example a liquid or solid oral dosage form. In alternate embodiments, the (+)-1-(3, 4-dichlorophenyl)-3-azabicyclo[3.1.0]hexane compound or a pharmaceutically acceptable salt thereof is contained in the kit in separate dosage forms for coordinate administration. An example of such a kit is a so-called blister pack. Blister packs are well-known in the packaging industry and are widely used for the packaging of pharmaceutical dosage forms (tablets, capsules and the like).

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list.

It is to be understood that this invention is not limited to the particular formulations, process steps, and materials disclosed herein as such formulations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The following examples illustrate certain aspects of the invention, but are not intended to limit in any manner the scope of the invention.

### Example I

### Comparison of Nicotine Sensitization in Alcohol-Preferring Rats Compared to Alcohol-Nonpreferring Rats

Sensitization of the neuro reward pathways are an enduring effect of a drug of abuse (Pierce and Kalivas, 1997). Animal sensitization models mimic drug addiction by creating behavioral effects indicative of chronic drug use in a more controlled environment (Schroeder et al., 2001; Miller et. al, 2001). This method is widely used to indirectly assess nicotine reinforcement because the effects of sensitization last even after the drug treatments have stopped. Behavioral sensitization models have been used to show that chronic exposure to drugs of abuse such as nicotine can have long-lasting effects on the neuromechanisms of reward pathways similar to the effects of nicotine addiction. The adaptations that occur in brain reward circuits in response to chronic potentiation of nicotine may model the development of the compulsive drug use that characterizes addiction (Ahmed et al., 2002).

Previous research has shown that alcohol-preferring (P) rats will self-administer nicotine at a greater rate than alcohol non-preferring rats (Le et al., 2006). In a test of open field locomotor activity, similar results to those previously reported were obtained.

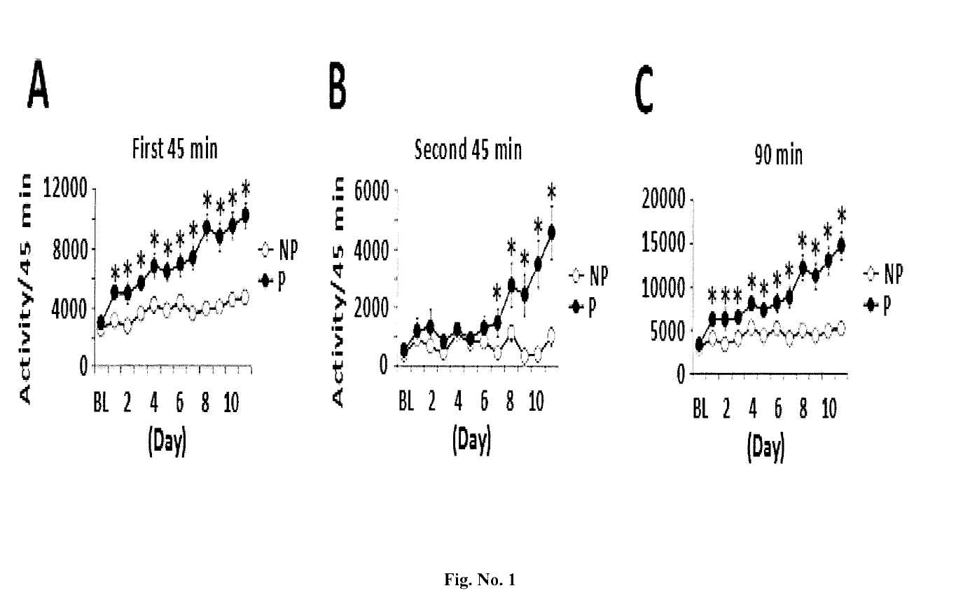

P and NP (alcohol-nonpreferring) rats were administered nicotine by subcutaneous injection of 0.4 mg/kg nicotine each day for 10 days. The rats were then immediately run for 90 minute locomotor sessions using an open-field activity monitoring cage (27×27×20.3 cm, Med Associates, Inc., St. Albans, Vt.). Activity counts represented by the number of infrared beam interruptions were recorded for each animal. As can be seen in FIG. 1, A-C, sensitization to nicotine occurs in P rats only. Notably, sensitization to nicotine during the 2nd 45 min of the experiment does not occur until day 7, which impacts the overall 90 min data. Together, these data suggest that P rats show an enhanced reward system in comparison to NP rats. (*P<0.001 by ANOVA)

### Example II

### Effect of (+)-1-(3, 4-dichlorophenyl)-3-azabicyclo[3.1.0]hexane on Nicotine Sensitization in Alcohol-Preferring Rats

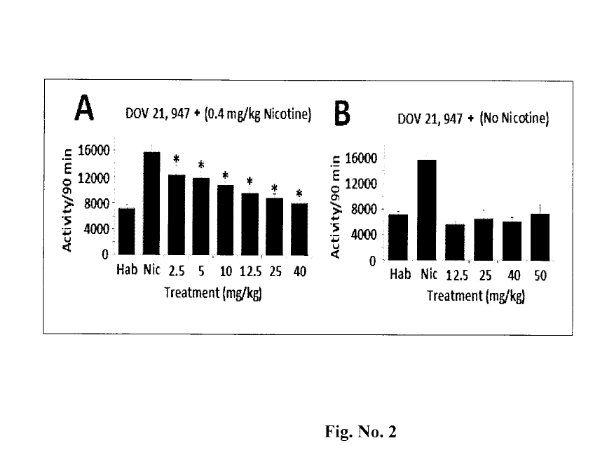

To evaluate the capacity of oral (+)-1-(3, 4-dichlorophenyl)-3-azabicyclo[3.1.0]hexane to block nicotine-induced sensitization in P rats, cohorts of P rats (n=6-9/group) were sensitized to nicotine as described in Example I. The rats were then divided into sixteen groups and given either vehicle, nicotine alone, 2.5 mg/kg (+)-1-(3, 4-dichlorophenyl)-3-azabicyclo[3.1.0]hexane, 5 mg/kg (+)-1-(3, 4-dichlorophenyl)-3-azabicyclo[3.1.0]hexane, 10 mg/kg (+)-1-(3, 4-dichlorophenyl)-3-azabicyclo[3.1.0]hexane, 25 mg/kg (+)-1-(3, 4-dichlorophenyl)-3-azabicyclo[3.1.0]hexane, or 40 mg/kg (+)-1-(3, 4-dichlorophenyl)-3-azabicyclo[3.1.0]hexane. Thirty minutes after administration of (+)-1-(3, 4-dichlorophenyl)-3-azabicyclo[3.1.0]hexane, half of the rats were given 0.4 mg/kg nicotine or vehicle. The rats were then immediately run for 90 minute locomotor sessions using an open-field activity monitoring cage (27×27×20.3 cm, Med Associates, Inc., St. Albans, Vt.). Activity counts represent the number of infrared beam interruptions were recorded for each animal. As can be seen in FIG. 2A, all doses of DOV 21,947 ((+)-1-(3, 4-dichlorophenyl)-3-azabicyclo[3.1.0]hexane) tested significantly reduced nicotine-induced increases in horizontal activity. (+)-1-(3, 4-dichlorophenyl)-3-azabicyclo[3.1.0]hexane did not alter locomotor activity when administered alone (FIG. 2B).

### Example III

### Effects of Nicotine on Reward Potentiating in Alcohol-Preferring Rats Exhibit in ICSS Paradigm

In addition to locomotor sensitization, intracranial self-stimulation (ICSS) was used to examine whether P rats, when compared to NP rats, showed an enhanced sensitivity to the reward potentiating effects of nicotine following sensitization as described in Example I. The rationale for use of the ICSS model as a measure to evaluate negative affective states is well known, particularly as it pertains to hypothesized depression-like symptoms due to drug-induced abstinence, even with nicotine (Markou et al., 1998; Koob, 2000; Cryan et al., 2002; 2003).

Rats were anesthetized with isoflurane and an electrode was implanted in the medial forebrain bundles as previously described (Eiler et al., 2005; 2007). Behavioral training and testing was conducted in standard operant chambers (Coulbourn Instruments, Allentown, Pa.) equipped with a removable lever enclosed in a sound attenuating cubicle as previously reported (Eiler et al., 2005; 2007). Four dependent variables were collected in the studies: (1) the frequency that corresponds with 50% of responding (EF₅₀), (2) the minimum/lowest frequency capable of maintaining brain stimulation reward (BSR), (3) the maximum frequency producing the highest rate of lever pressing throughout the BSR session, and (4) the total number of lever presses produced during a 20 minute session.

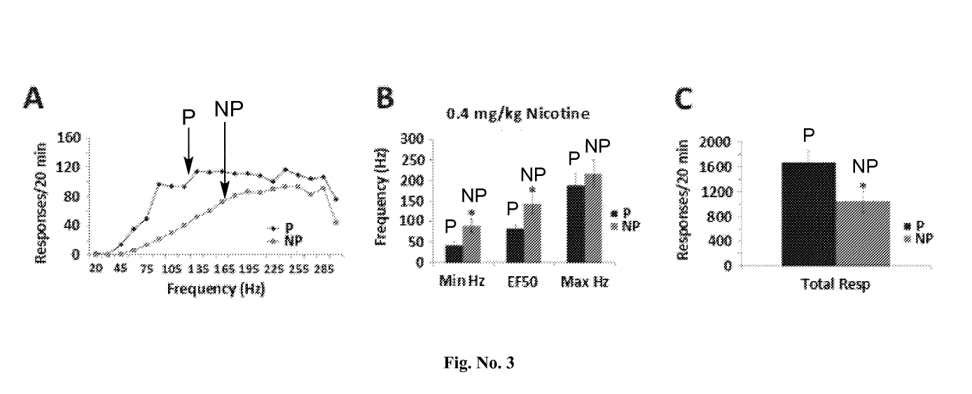

After surgical implantation, rats were trained on a continuous reinforcement (FR1) schedule, then decreased to a frequency of reinforcement after every six correct responses (FR6) until stabilization. After training, the rats were sensitized with nicotine (0.4 mg/kg daily) using the Kelley 10-day sensitization model as described in Example 1 (rats were treated with either nicotine (0.4 mg/ml/kg) or saline once per day for 10 days in a test environment distinct from their home cages (Schroeder et al., 2001). On day 11, rats (n=8/group) were injected with nicotine subcutaneously (0.4 mg/kg) and run for a 20 minute, FR6 ICSS session with a 300-20 Hz descending frequency schedule at the current that elicits the maximum number of responses. As shown in FIG. 3, there is significant separation of P vs. NP rats responding as shown by the rate-frequency function (FIG. 3A), minimum frequency, EF₅₀, maximum frequency (FIG. 3B), and total responding (FIG. 3C). Therefore, P, but not NP, rats exhibit an enhanced sensitivity to the reward potentiating effects of nicotine in ICSS following sensitization. The fact that P rats show an increased potentiation to nicotine is not due to increased sensitivity to ICSS itself in the medial forebrain bundle (MFB) as previous data has shown that naive P rats compared to NP rats do not differ in their sensitivity to ICSS (Eiler et al., 2007; 2005).

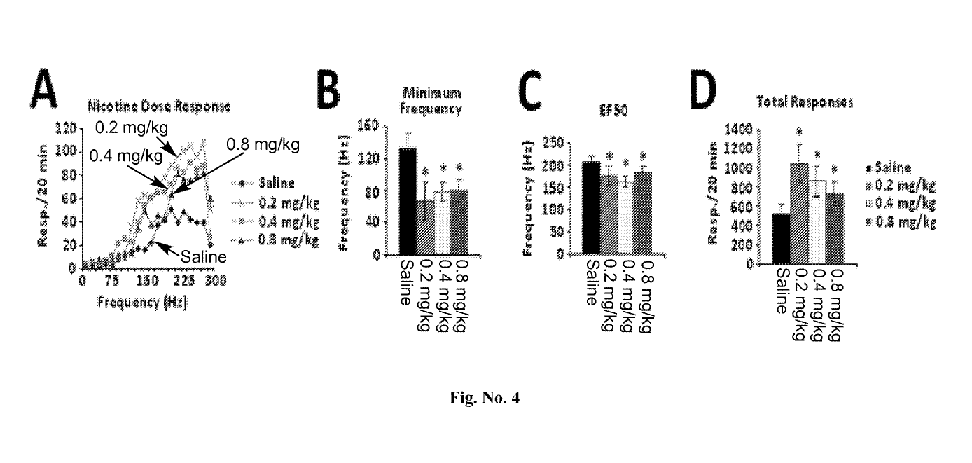

Following the initial ICSS experiments, the effects of varied doses of nicotine were examined in the ICSS paradigm. Animals were prepared and trained as described above. After training, the rats were sensitized with nicotine (0.4 mg/kg daily) using the Kelley 10-day sensitization model as described in Example I (rats were treated with either nicotine (0.4 mg/ml/kg) or saline once per day for 10 days in a test environment distinct from their home cages (Schroeder et al., 2001). On day 11, the rats (n=8/group) were given 0.2-0.8 mg/kg of nicotine SQ. The rats were then run for a 20 min FR6 ICSS session with a 300-20 Hz descending frequency schedule at the current that elicits the maximum number of responses as determined in the previous experiment. As shown in FIG. 4, all nicotine doses elicited higher responding than did saline (FIGS. 4A, 4D). The reward potentiating effects of nicotine were observed with all 3 doses via reduction in EF50 (FIG. 4C) and minimum frequency (FIG. 4B). There was no dose response observed.

### Example IV

### Effect of (+)-1-(3, 4-dichlorophenyl)-3-azabicyclo[3.1.0]hexane on Nicotine Potentiation of ICSS in Alcohol-Preferring Rats

The effect of (+)-1-(3, 4-dichlorophenyl)-3-azabicyclo[3.1.0]hexane on the nicotine facilitation of ICSS was examined in P rats. Rats were anesthetized with isoflurane and an electrode was implanted in the medial forebrain bundles as previously described (Eiler et al., 2005; 2007). Behavioral training and testing was conducted in standard operant chambers (Coulbourn Instruments, Allentown, Pa.) equipped with a removable lever enclosed in a sound attenuating cubicle as previously reported (Eiler et al., 2005; 2007). Four dependent variables were collected in the studies: (1) the frequency that corresponds with 50% of responding (EF₅₀), (2) the minimum/lowest frequency capable of maintaining brain stimulation reward (BSR), (3) the maximum frequency producing the highest rate of lever pressing throughout the BSR session and (4) the total number of lever presses produced during a 20 minute session.

After surgical implantation, rats were trained on a continuous reinforcement (FR1) schedule, then decreased to a frequency of reinforcement after every six correct responses (FR6) until stabilization. After training, the rats were sensitized with nicotine (0.4 mg/kg daily) using the Kelley 10-day sensitization model as described in Example I (rats were treated with either nicotine (0.4 mg/ml/kg) or saline once per day for 10 days in a test environment distinct from their home cages (Schroeder et al., 2001).

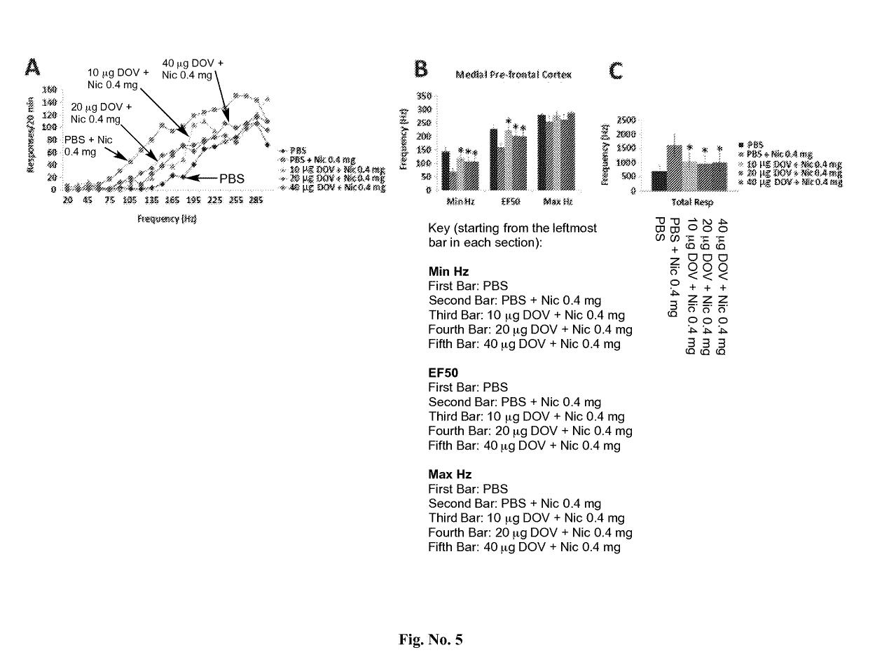

After sensitization, three of the nicotine cohorts of P rats (n=8) were given (+)-1-(3, 4-dichlorophenyl)-3-azabicyclo[3.1.0]hexane (10, 20 or 40 μg) into the medial prefrontal cortex (mPC) prior to the daily administration of nicotine (0.4 mg/ml/kg). When (+)-1-(3, 4-dichlorophenyl)-3-azabicyclo[3.1.0]hexane was administered immediately prior to nicotine, it significantly reduced the nicotine facilitation on ICSS as evidenced by a right-ward shift from the nicotine rate frequency function (FIG. 5A), attenuation of the nicotine reduction of minimum frequency and EF₅₀ (FIG. 5B) and elevation in total nicotine responding (FIG. 5C), but no dose response was apparent. These data suggest that the mPC may be a brain substrate in which (+)-1-(3, 4-dichlorophenyl)-3-azabicyclo[3.1.0]hexane is capable of antagonizing nicotine's actions.

### Example V

### Effect of Withdrawal on ICSS Paradigm in Alcohol-Preferring Rats

Rats were anesthetized with isoflurane and an electrode was implanted in the medial forebrain bundles as previously described (Eiler et al., 2005; 2007). Behavioral training and testing was conducted in standard operant chambers (Coulbourn Instruments, Allentown, Pa.) equipped with a removable lever enclosed in a sound attenuating cubicle as previously reported (Eiler et al., 2005; 2007). Four dependent variables were collected in the studies: (1) the frequency that corresponds with 50% of responding (EF₅₀), (2) the minimum/lowest frequency capable of maintaining brain stimulation reward (BSR), (3) the maximum frequency producing the highest rate of lever pressing throughout the BSR session and (4) the total number of lever presses produced during a 20 minute session.

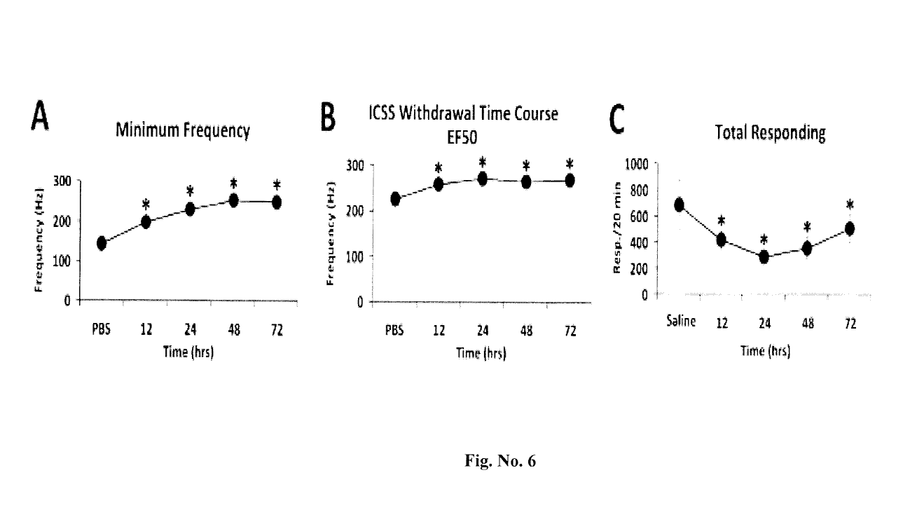

After surgical implantation, rats were trained on a continuous reinforcement (FR1) schedule, and then decreased to a frequency of reinforcement after every six correct responses (FR6) until stabilization. After training, the rats were sensitized with nicotine (0.4 mg/kg daily) for 14 days. 12 hours after the last dose of nicotine, animals were run for a 20 min FR-6 ICSS sessions. ICSS sessions were run every 12 hours (12 hrs, 24 hrs, 36 hrs, 48 hrs, 60 hrs and 72 hrs) for 72 hours. As shown in FIG. 6, there was a time dependent increase of minimum frequency capable of maintaining BSR (FIG. 6A), EF₅₀ (FIG. 6B) and a marked reduction in total ICSS responding (FIG. 6C) during the withdrawal period. These data illustrate the negative affective states (e.g. anhedonia) created by nicotine withdrawal.

### Example VI

### Effect of (+)-1-(3, 4-dichlorophenyl)-3-azabicyclo[3.1.0]hexane on Nicotine-Induced Withdrawal Effects in Alcohol-Preferring Rats Using ICSS Paradigm

The effect of (+)-1-(3, 4-dichlorophenyl)-3-azabicyclo[3.1.0]hexane on nicotine withdrawal and ICSS was examined in P rats. Rats were anesthetized with isoflurane and an electrode was implanted in the medial forebrain bundles as previously described (Eiler et al., 2005; 2007). Behavioral training and testing was conducted in standard operant chambers (Coulbourn Instruments, Allentown, Pa.) equipped with a removable lever enclosed in a sound attenuating cubicle as previously reported (Eiler et al., 2005; 2007). Four dependent variables were collected in the studies: (1) the frequency that corresponds with 50% of responding (EF₅₀), (2) the minimum/lowest frequency capable of maintaining brain stimulation reward (BSR), (3) the maximum frequency producing the highest rate of lever pressing throughout the BSR session and (4) the total number of lever presses produced during a 20 minute session.

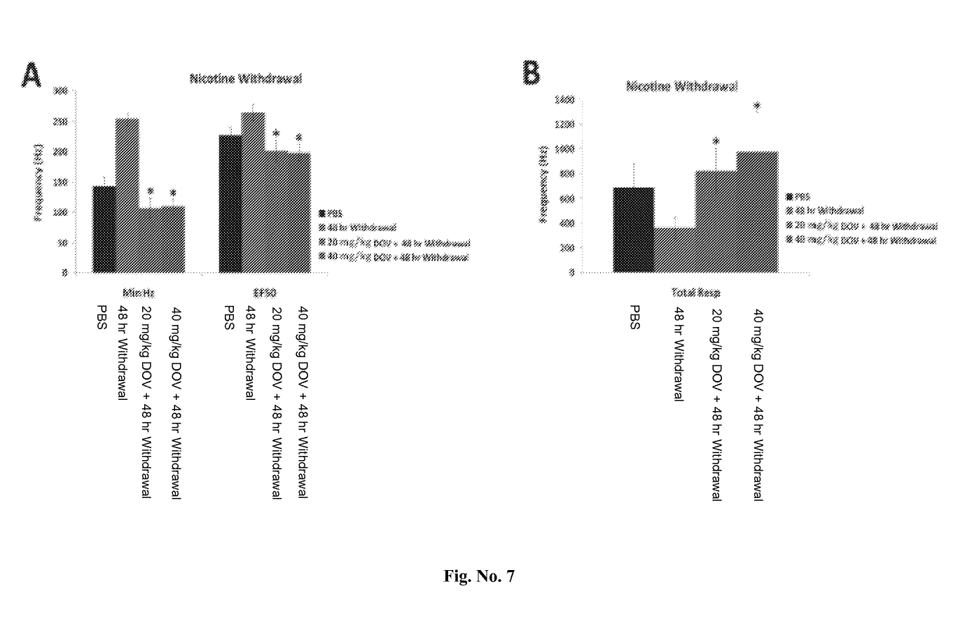

After surgical implantation, rats were trained on a continuous reinforcement (FR1) schedule, and then decreased to a frequency of reinforcement after every six correct responses (FR6) until stabilization. After training, the rats were sensitized with nicotine (0.4 mg/kg daily) for 14 days. Rats were then given either PBS, 20 mg/kg of (+)-1-(3, 4-dichlorophenyl)-3-azabicyclo[3.1.0]hexane or 40 mg/kg of (+)-1-(3, 4-dichlorophenyl)-3-azabicyclo[3.1.0]hexane. After 48 hours, the animals were run for a 20 min FR-6 ICSS session. As shown in FIG. 7, oral doses of (+)-1-(3, 4-dichlorophenyl)-3-azabicyclo[3.1.0]hexane (20 and 40 mg/kg) produced a profound reduction on nicotine elevation of the reward threshold at 48 hours nicotine abstinence on the minimum frequency and EF50 parameters (FIG. 7A). (+)-1-(3, 4-dichlorophenyl)-3-azabicyclo[3.1.0]hexane also elevated the reduction in responding (FIG. 7B) produced by the 48 hour withdrawal period. These data indicate oral (+)-1-(3, 4-dichlorophenyl)-3-azabicyclo[3.1.0]hexane is capable of attenuating nicotine-induced abstinence effects as measured by elevations in ICSS responding thresholds, indicative of attenuation of negative affective states produced by nicotine withdrawal.

### Example VII

### Effect of (+)-1-(3, 4-dichlorophenyl)-3-azabicyclo[3.1.0]hexane on Nicotine Withdrawal on Alcohol-Preferring Rat Forced Swim Test Paradigms

The forced swim test (FST) is an animal model of depression in which nicotine withdrawal causes increased immobility (i.e. despair) in the rodents. The FST accordingly may be used as an animal model to evaluate negative affective states during nicotine withdrawal, which may emulate “depressive-like” behavior in human nicotine addicts. An emerging body of research has shown that withdrawal from drugs of abuse such as morphine (Anraku et al., 2001), phencyclidine (Noda et al., 2000) amphetamine (Kokkinidis et al., 1986; Cryan et al., 2002), cocaine (Pliakas et al., 2001), and nicotine (Paterson and Markou, 2007) increase immobility in the forced swim test (FST) in rodents.

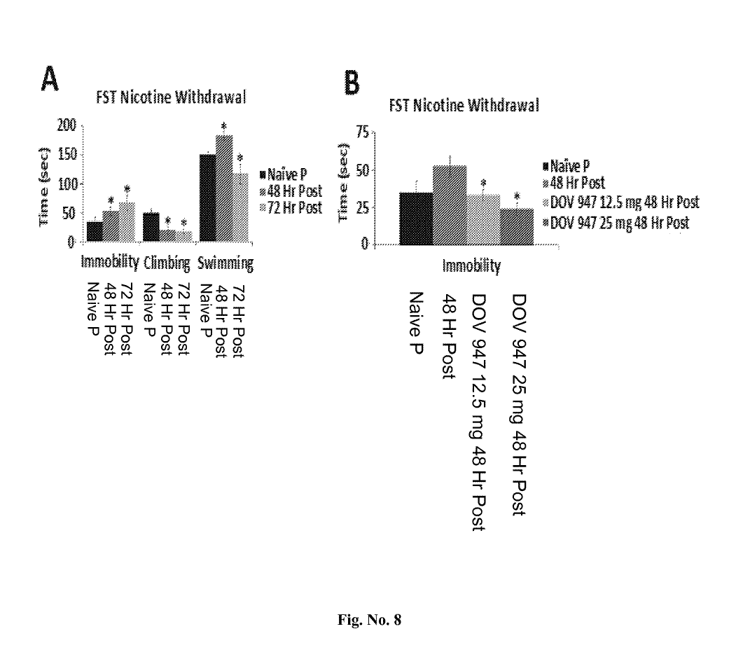

P rats were administered nicotine by subcutaneous injection of 0.4 mg/kg nicotine each day for 14 days and then deprived of nicotine. Lucki's modified FST (Cryan et al., 2002) was performed using immobility, climbing and swimming every twelve hours following the last dose of nicotine (12 hrs, 24 hrs, 36 hrs, 48 hrs, 60 hrs and 72 hrs) for 72 hours. As shown in FIG. 8, nicotine abstinence results in a significant elevation in immobility at 48 and 72 hours (FIG. 8A). Reduction in climbing and swimming were also observed. Oral (+)-1-(3, 4-dichlorophenyl)-3-azabicyclo[3.1.0]hexane (12.5 or 25 mg/kg) administered at 48 hours significantly attenuated the nicotine-induced increases in immobility (FIG. 8B). Together the data suggest that the FST can be used as a model to observe “depressive-like” symptoms following nicotine abstinence and that oral (+)-1-(3, 4-dichlorophenyl)-3-azabicyclo[3.1.0]hexane can be used to attenuate these effects.

### Example VIII

### Effect of (+)-1-(3, 4-dichlorophenyl)-3-azabicyclo[3.1.0]hexane on Binding of Ligands to Nicotinic Receptors

To examine the possibility that a (+)-1-(3, 4-dichlorophenyl)-3-azabicyclo[3.1.0]hexane may have effects mediated via direct actions on nicotinic receptors, its affinity was determined for α7 and α4β2 nicotinic receptors in radioligand binding assays. The assessment was conducted by Cerep, Inc. (Poitiers, France). Inhibition of binding to α4β2 nicotinic receptors in SH-SYSY cells was determined using the radioligand [³H]cytosine at 0.6 nM concentration according to the method of Gopalakrishnan et al. (1996). Incubation was for 120 minutes at 4° C. and non-specific binding was determined with 10 μM nicotine. Inhibition of binding to α7 nicotinic receptors in SH-SYSY cells was determined using [¹²⁵I]α-bungarotoxin (0.05 nM) according to the method of Sharples et al. (2000). Incubation was 120 minutes at 37° C. and non-specific binding was determined using α-bungarotoxin (1 μM). Specific binding of the radioligands was determined by scintillation spectrometry technology.

As shown in Table 5, there was no appreciable effect of a (+)-1-(3, 4-dichlorophenyl)-3-azabicyclo[3.1.0]hexane at concentrations from 100 to 10,000 nM on binding of radioligands to either α7 or α4β2 nicotinic receptors, suggesting that the effects of (+)-1-(3, 4-dichlorophenyl)-3-azabicyclo[3.1.0]hexane are not mediated by direct interaction with nicotinic receptors.

TABLE 5
Affinity of amitifadine ((+)-1-(3,4-dichlorophenyl)-
3-azabicyclo[3.1.0]hexane) for α7 and
α4β2 nicotinic receptors

| | Concentration of (+)-1-(3,4-dichlorophenyl)-3- | | |
|---|---|---|---|
| | azabicyclo[3.1.0]hexane, nM | | |
| | 100 | 1000 | 10000 |
| Nicotinic receptor | % Inhibition of binding | | |
| α4β2 | 17 | 14 | 13 |
| α7 | 13 | 6 | −9 |

### Example IX

### Effect of (+)-1-(3, 4-dichlorophenyl)-3-azabicyclo[3.1.0]hexane on Nicotine Self-Administration in Female Rats

A study was undertaken to determine whether (+)-1-(3, 4-dichlorophenyl)-3-azabicyclo[3.1.0]hexane would decrease nicotine self-administration at doses that do not cause adverse side effects. Adult female Sprague-Dawley rats were trained to self-administer nicotine IV and were given acute doses of (+)-1-(3, 4-dichlorophenyl)-3-azabicyclo[3.1.0]hexane in a repeated measures counterbalanced design.

Young adult female (n=10) Sprague-Dawley rats (Taconic Lab, Germantown, N.Y., USA) were used in the study. Animals were individually housed in a temperature controlled vivarium room located adjacent to the nicotine self-administration testing room. Animals were maintained on a 12:12 reverse light-dark cycle so that experimental sessions occurred during the active part of the rats' diurnal cycle. Animals were given ad lib access to water at all times excluding experimental sessions, and were fed daily 20-30 minutes after the completion of their experimental session in an amount to keep the rats at a lean healthy weight. This study was conducted under a protocol approved by the Duke University Institutional Animal Care and Use Committee in accordance with USDA regulations.

Nicotine bitartrate solutions were prepared in isotonic sterile saline. The dose used for self-administration (0.03 mg/kg/infusion) was calculated as a function of the nicotine free base weight. The pH of the nicotine solution was adjusted to 7.0 using NaOH and the solution was filtered in a Nalgene filter (Nalgene Nunc International, Rochester, N.Y., USA) for sterilization. Between sessions all nicotine was kept in a dark refrigerator.

(+)-1-(3, 4-dichlorophenyl)-3-azabicyclo[3.1.0]hexane solutions were also prepared in sterile water for doses of 5, 10 and 30 mg/kg (po). Water was used as the control. The volume of oral gavage was 1 ml/kg given 30 minutes before testing. The drug doses and control vehicle (sterile water) were administered in a repeated measures counterbalanced design with at least two days between consecutive injections. The entire dose-effect function was run twice.

Before the start of nicotine self-administration sessions, all animals were trained to lever press for food reinforcement in a standard dual-lever operant chamber (Med Associates, St. Albans, Vt., USA). Each chamber was equipped with: two levers, two cue lights located directly above each lever, a house light, and a tone generator. After lever pressing was established, animals experienced three sessions of lever pressing for food under a fixed ratio (FR) 1 schedule of reinforcement. Following the completion of their final training session with food reinforcement, animals were anesthetized with a mixture of ketamine (60 mg/kg) and dormitor (15 mg/kg) and a catheter (Strategic Application Inc., Libertyville, Ill., USA) was implanted into their jugular vein. The jugular catheter was attached to a harness that could be tethered to the infusion pump during experimental sessions.

Following surgery, animals experienced 5 experimental sessions where a correct lever press resulted in the delivery of a nicotine infusion (0.03 mg/kg/infusion) on a fixed ratio (FR) 1 schedule of reinforcement, and the activation of a feedback tone for 0.05 s. Each infusion was followed by a one-minute period where the cue lights went out, the house light came on and correct responses were recorded but not reinforced. After the initial 5 sessions of nicotine self-administration, the rats were tested for effects of acute (+)-1-(3, 4-dichlorophenyl)-3-azabicyclo[3.1.0]hexane on nicotine self-administration in a repeated measures counterbalanced design with all of the rats receiving all of the doses two times in separate phases. The sessions were 45 minutes long.

The catheters were flushed daily, before the experimental sessions, with a 100 U/ml heparinized saline solution. After the completion of each test session nicotine remaining in the port was removed and a 0.3 ml sterile lock solution containing 500 U/ml of heparinized saline and 8 mg/ml of gentamicin was infused (American Pharmaceutical Partners, Schaumberg, Ill., USA).

The data were evaluated with a repeated measures analysis of variance. (+)-1-(3, 4-dichlorophenyl)-3-azabicyclo[3.1.0]hexane dose was the principal within-subjects factors. Blocks within session and repeated testing were also within subject factors. An alpha level of p<0.05 was used to determine statistical significance. Significant interactions were followed by tests of the simple main effects. Planned comparisons were used to assess the significance each of the (+)-1-(3, 4-dichlorophenyl)-3-azabicyclo[3.1.0]hexane dose to the control vehicle.

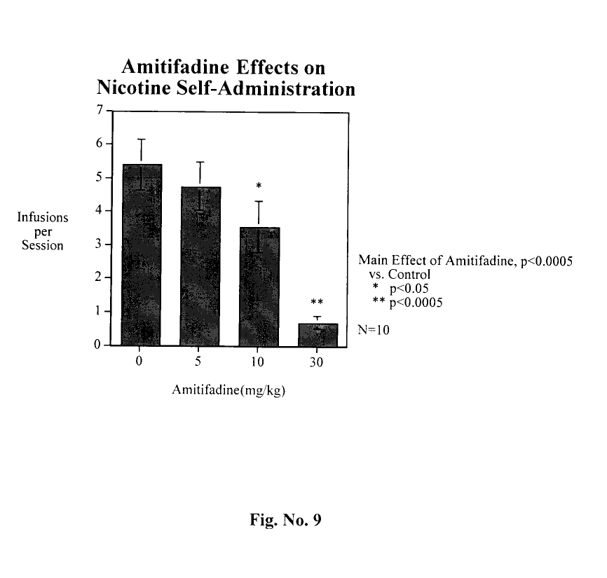

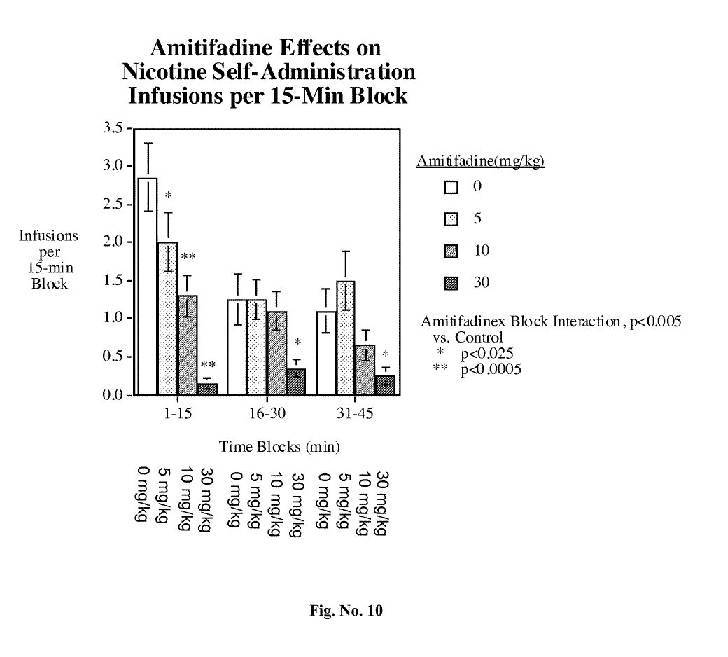

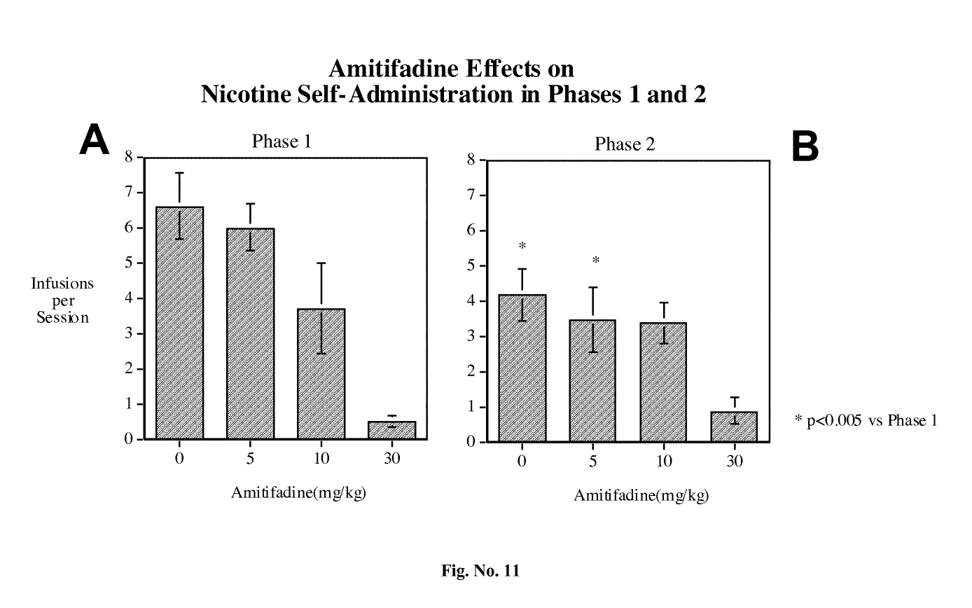

Acutely administered (+)-1-(3, 4-dichlorophenyl)-3-azabicyclo[3.1.0]hexane significantly (F(3,27)=13.54, p<0.0005) reduced nicotine self-administration. As shown in FIG. 9, the 10 mg/kg (p<0.05) and the 30 mg/kg (p<0.0005) doses of (+)-1-(3, 4-dichlorophenyl)-3-azabicyclo[3.1.0]hexane each significantly reduced nicotine self-administration relative to control treatment averaged over the 45-minute sessions. There was a significant (F(6,54)=4.05, p<0.005) interaction of (+)-1-(3, 4-dichlorophenyl)-3-azabicyclo[3.1.0]hexane and 15-minute time block within the 45-minute session (FIG. 10). Tests of the simple main effects of the (+)-1-(3, 4-dichlorophenyl)-3-azabicyclo[3.1.0]hexane dose effects at each time block showed that the high 30 mg/kg dose caused significant (p<0.0005) reductions during all of the time blocks of the session. Both the 5 mg/kg (p<0.025) and 10 mg/kg (p<0.0005) doses caused significant decreases in nicotine self-administration during the first 15-minute block, but not during the later parts of the session as the control rates of nicotine self-administration decreased relative to the first 15-minute period. (+)-1-(3, 4-dichlorophenyl)-3-azabicyclo[3.1.0]hexane was administered in a repeated measures counterbalanced design twice. There was a significant interaction of (+)-1-(3, 4-dichlorophenyl)-3-azabicyclo[3.1.0]hexane and test phase (F(3,27)=3.59, p<0.05). As shown in FIG. 11, during the second test phase nicotine self-administration for the 0 and 5 mg/kg conditions were significantly (p<0.005) lower than during the first test phase. Nicotine self-administration with the 10 and 30 mg/kg conditions did not differ between the two test phases. Thus, (+)-1-(3, 4-dichlorophenyl)-3-azabicyclo[3.1.0]hexane blunted the initial flurry of nicotine self-administration during the first 15 minutes of nicotine self-administration. Overall, (+)-1-(3, 4-dichlorophenyl)-3-azabicyclo[3.1.0]hexane significantly reduced nicotine self-administration at several doses.

In other aspects of the invention, exemplary formulations of (+)-1-(3, 4-dichlorophenyl)-3-azabicyclo[3.1.0]hexane were produced and evaluated for use within the methods and compositions of the invention, as briefly described in Examples X-XIII below.

### Example X

### Preparation of 50 Mg. (+)-1-(3,4-dichlorophenyl)-3-azabicyclo[3.1.0]hexane HCl Tablet

Immediate release tablets containing 50 mg of the HCl salt of (+)-1-(3,4-dichlorophenyl)-3-azabicyclo[3.1.0]hexane are prepared using the following ingredients. In Table 6 below the “% composition” is the % by weight of the ingredient based upon the total weight of the composition.

TABLE 6
(+)-1-(3,4-dichlorophenyl)-3-azabicyclo[3.1.0]hexane HCl Tablets

| Material | % Composition | Mg/tablet |
|---|---|---|
| (+)-1-(3,4-dichlorophenyl)-3- | 22.22 | 50.00 |
| azabicyclo[3.1.0]hexane (HCl salt) | | |
| Dibasic Calcium Phosphate, NF | 36.00 | 81.00 |
| Microcrystalline cellulose, NF | 36.00 | 81.00 |
| Croscarmellose Sodium, NF | 4.44 | 10.00 |
| Colloidal Silicon Dioxide, NF | 0.67 | 1.50 |
| Magnesium Stearate, NF (veg grade) | 0.67 | 1.50 |

Each tablet may also be coated with 6.00 mg of Opadry II White (85F18422).

### Example XI

### Preparation of 50 Mg. (+)-1-(3,4-dichlorophenyl)-3-azabicyclo[3.1.0]hexane HCl Capsule

Immediate release capsules containing 50 mg of the HCl salt of (+)-1-(3,4-dichlorophenyl)-3-azabicyclo[3.1.0]hexane are prepared using the following ingredients. In Table 7 below the “% composition” is the % by weight of the ingredient based upon the total weight of the composition.

TABLE 7
(+)-1-(3,4-dichlorophenyl)-3-azabicyclo[3.1.0]hexane
HCl Capsules

| Material | % Composition | Mg/tablet |
|---|---|---|
| (+)-1-(3,4-dichlorophenyl)-3- | 24.39 | 50.00 |
| azabicyclo[3.1.0]hexane (HCl salt) | | |
| Mannitol, Spray Dried, USP | 72.28 | 148.16 |
| Talc, USP | 2.63 | 5.40 |
| Magnesium Stearate, NF | 0.70 | 1.44 |

The ingredients are encapsulated in a white opaque capsule #3.

### Example XII

### Preparation of 100 Mg. (+)-1-(3,4-dichlorophenyl)-3-azabicyclo[3.1.0]hexane HCl Extended Release Tablet

Once per day, extended release tablets containing 100 mg of the HCl salt of (+)-1-(3,4-dichlorophenyl)-3-azabicyclo[3.1.0]hexane are prepared using the following ingredients. In Table 8 below the “% composition” is the % by weight of the ingredient based upon the total weight of the composition.

TABLE 8
(+)-1-(3,4-dichlorophenyl)-3-azabicyclo[3.1.0]hexane
HCl Extended Release Tablets

| Material | % Composition | Mg/tablet |
|---|---|---|
| (+)-1-(3,4-dichlorophenyl)-3- | 28.6 | 100.00 |
| azabicyclo[3.1.0]hexane (HCl salt) | | |
| Methocel Premium CR | 30.0 | 105.00 |
| MicroCrystalline Cellulose | 20.4 | 71.50 |
| Starch 1500 | 20.0 | 70.00 |
| Colloidal Silicon Dioxide | 0.5 | 1.75 |
| Magnesium Stearate | 0.5 | 1.75 |

The tablets are manufactured by direct compression into ⅜″ round, standard biconvex tablets. The microcrystalline cellulose used is 90 micron grade. A pregelatinized starch is used in the tablets. The Methocel Premium CR can be Methocel K4 or Methocel K100. Each tablet may also be coated, such as with 5.5% Opadry II White (85F18422).

### Example XIII

### Dissolution of 100 Mg. (+)-1-(3,4-dichlorophenyl)-3-azabicyclo[3.1.0]hexane HCl Extended Release Tablet

Dissolution testing of tablets manufactured according to Example XII was performed on tablets containing either Methocel K4 or K100, and tablets were either coated or uncoated. Dissolution Testing was performed using USP Apparatus 2, 50 rpm, 900 ml water, 37° C.

TABLE 9
Dissolution Testing of (+)-1-(3,4-
dichlorophenyl)-3-azabicyclo[3.1.0]hexane
HCl Extended Release Tablets

| | K4M | K4M | K100M | K100M |
|---|---|---|---|---|
| Time | uncoated | coated | uncoated | coated |
| (Hours) | % Dissolved | % Dissolved | % Dissolved | % Dissolved |
| 0.5 | 11.11 | 0.26 | 10.13 | 0.38 |
| 1 | 16.77 | 0.30 | 14.92 | 0.20 |
| 2 | 23.79 | 1.78 | 22.71 | 0.38 |
| 4 | 35.35 | 9.36 | 34.98 | 1.80 |
| 6 | 43.14 | 19.91 | 45.49 | 6.66 |
| 8 | 52.24 | 30.95 | 53.30 | 14.39 |
| 10 | 59.22 | 40.32 | 59.99 | 23.27 |
| 12 | 67.67 | 49.85 | 66.98 | 32.78 |
| 25 | 104.44 | 83.32 | 78.31 | 68.43 |

The results of the dissolution testing depicted in Table 9 above confirm that a slow dissolution profile was achieved for an extended release tablet of (+)-1-(3,4-dichlorophenyl)-3-azabicyclo[3.1.0]hexane, HCl salt form. The results further show that the (+)-1-(3,4-dichlorophenyl)-3-azabicyclo[3.1.0]hexane was released at or nearly at a continuous or nearly same rate over 24 hours, and in particular was released at a continual or nearly continual/same rate between 2-12 hours (120-720 minutes). The amount of (+)-1-(3,4-dichlorophenyl)-3-azabicyclo[3.1.0]hexane released over 24 hours was from about 65% (68% in the K100M coated example) to 100%, and overall averaged about 83% released, with 3 samples of tablets having released 78, 83, and 100% of the (+)-1-(3,4-dichlorophenyl)-3-azabicyclo[3.1.0]hexane initially contained therein. The amount of (+)-1-(3,4-dichlorophenyl)-3-azabicyclo[3.1.0]hexane released at 12 hours following administration was from about 33% to about 70%.

Although the foregoing invention has been described in detail by way of example for purposes of clarity of understanding, persons of ordinary skill in the art will understand that certain changes and modifications may be practiced within the scope of the appended claims which are presented by way of illustration not limitation. In this context, the invention is not limited to the particular formulations, processes, and materials disclosed herein, as such formulations, process steps, and materials may vary somewhat. Also, the terminology employed herein is used for describing particular embodiments only, and is not intended to be limiting of the invention embodied in the claims. Various publications and other reference information have been cited within the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

### REFERENCES

Ahmed S H, Kenny P J, Koob G F, Markou A. Neurobiological evidence for hedonic allostasis associated with escalating cocaine use. Nat Neurosci. 2002 July; 5(7):625-6.
Anraku T, Ikegaya Y, Matsuki N, Nishiyama N. Withdrawal from chronic morphine administration causes prolonged enhancement of immobility in rat forced swimming test. Psychopharmacology (Berl). 2001 September; 157(2):217-20.
Astrup A, Madsbad S, Breum L, Jensen T J, Kroustrup J P, Larsen T M. Effect of Tesofensine on Bodyweight Loss, Body Composition, and Quality of Life in Patients: a Randomised, Double-Blind, Placebo-Controlled Trial. Lancet 2008, 372:1906-13.
Beck, A. T., R. A Steer (1988) Manual for Beck Hopelessness Scale. Psychological Corp., Harcourt Brace Jovanovich San Antonio, Tex.
Beck, A. T., R. A Steer (1991) Manual for Beck Scale For Suicide Ideation. San Antonio, Tex.: Psychological Corporation.
Benowitz N L, Peng M W (2000) Non-nicotine pharmacotherapy for smoking cessation —Mechanisms and prospects. CNS Drugs 13: 265-285.
Breslau N (1995) Psychiatric comorbidity of smoking and nicotine dependence. Behav Genet 25: 95-101.
Coric, Vladimir et al., Sheehan Suicidality Tracking Scale (Sheehan-STS): Preliminary Results from a Multicenter Clinical Trial in Generalized Anxiety Disorder. Psychiatry (Edgmont (Pa.: Township)) 2009 6 (1): 26-31.
Cryan J F, Hoyer D, Markou A. Withdrawal from chronic amphetamine induces depressive-like behavioral effects in rodents. Biol Psychiatry. 2003 Jul. 1; 54(1):49-58.
Cryan J F, Markou A, Lucki I. Assessing antidepressant activity in rodents: recent developments and future needs. Trends Pharmacol Sci. 2002 May; 23(5):238-45.
DeLorenzo C, Lichenstein S, Schaefer K, Dunn J, Marshall R, Organisak L, Kharidia J, Robertson B, Mann J J, Parsey R V. SEP-225289 Serotonin and Dopamine Transporter Occupancy: a PET Study. J. Nucl. Med. 2011, 52:1150-1155.
Di Chiara G, Bassareo V, Fenu S, De Luca M A, Spina L, Cadoni C, Acquas E, Carboni E, Valentini V, Lecca D (2004) Dopamine and drug addiction: The nucleus accumbens shell connection. Neuropharmacology 47 Suppl 1: 227-241.
Duboc, Bruno, http://thebrain.mcgill.ca/flash/i/i_03/i_03_m/i_03_m_par/i_03_m_par_heroine.html Canadian Institutes of Health Research: Institute of Neurosciences, Mental Health and Addiction (2002).
Dudas M M, George T P (2005) Non-nicotine pharmacotherapies for nicotine dependence. Essential Psychopharmacology 6: 158-172.
Eiler W J 2nd, Hardy L 3rd, Goergen J, Seyoum R, Mensah-Zoe B, June H L. Responding for brain stimulation reward in the bed nucleus of the stria terminalis in alcohol-preferring rats following alcohol and amphetamine pretreatments. Synapse. 2007 November; 61(11):912-24.
Eiler W J 2nd, Woods J E 2nd, Masters J, McKay P F, Hardy L 3rd, Goergen J J, Mensah-Zoe B, Cook J B, Johnson N J, June H L. Brain stimulation reward performance and sucrose maintained behaviors in alcohol-preferring and -nonpreferring rats. Alcohol Clin Exp Res. 2005 April; 29(4):571-83.
FDA New Release, Oct. 8, 2010, http://www.fda.gov/NewsEvents/Newsroom/PressAnnouncements/2010/ucm228812.htm.
First, Michael B., Williams, Janet B. W., Spitzer, Robert L., and Gibbon, Miriam: Structured Clinical Interview for DSM-IV-TR Axis I Disorders, Clinical Trials Version (SCID-C T). New York: Biometrics Research, New York State Psychiatric Institute, 2007.
Frishman W H (2007) Smoking cessation pharmacotherapy—nicotine and non-nicotine preparations. Preventive Cardiology 10: 10-22.
Frishman W H, Mitta W, Kupersmith A, Ky T (2006) Nicotine and non-nicotine smoking cessation pharmacotherapies. Cardiology in Review 14: 57-73.
Glassman A H, Stetner F, Walsh B T, Raizman P S, Fleiss J L, Cooper T B, Covey L S (1988) Heavy smokers, smoking cessation, and clonidine: Results of a double-blind, randomized trial. Journal of the American Medical Association 259: 2863-2866.
Golembiowska K, Kowalska M, Bymaster F P (2012) Effects of the triple reuptake inhibitor amitifadine on extracellular levels of monoamines in rat brain regions and on locomotor activity. Synapse In press.
Gopalakrishnan M, Monteggia L M, Anderson D J, Molinari E J, Piattoni-Kaplan M, Donnelly-Roberts D, Arneric S P, Sullivan J P (1996) Stable expression, pharmacologic properties and regulation of the human neuronal nicotinic acetylcholine alpha 4 beta 2 receptor. Journal of Pharmacology and Experimental Therapeutics 276: 289-297.
Gopalakrishnan M, Monteggia L M, Anderson D J, Molinari E J, Piattoni-Kaplan M, Donnelly-Roberts D, Arneric S P, Sullivan J P. Stable expression, pharmacologic properties and regulation of the human neuronal nicotinic acetylcholine alpha 4 beta 2 receptor. J Pharmacol Exp Ther. 1996 January; 276(1):289-97.
Graff, Ole et al. Results of two double blind Placebo and Active-controlled Studies of GSK372475, a Triple Monoamine Reuptake Inhibitor, in the Treatment of Major Depressive Disorder. (ACNP 2009)
Guha M, Heier A, Price S, Bielenstein M, Caccese R G, Heathcote D I, Simpson T R, Stong D B, Bodes E. Assessment of Biomarkers of Drug-induced Kidney Injury in Cynomolgus Monkeys Treated with a Triple Re-uptake Inhibitor. Toxicol. Sci. 2011, 120:269-83.
Hamilton M. A rating scale for depression. J. Neurol. Neurosurg. Psychiat., 1960, 23, 56.
Hamilton M. Development of a rating scale for primary depressive illness. Br J Soc Clin Psychol. 1967; 6(4):278-96
Hamilton, M. (1959). The assessment of anxiety states by rating. British Journal of Medical Psychology, 32, 50-55.
Hatsukami D K, Stead L F, Gupta P C (2008) Tobacco addiction. Lancet 371: 2027-2038
Herber D, Carpenter C L, Addictive genes and the relationship to obesity and inflammation Mol Neurobiol. 2011 October; 44(2):160-5. Epub 2011 Apr. 19.
Hitsman B, Pingitore R, Spring B, Mahableshwarkar A, Mizes J S, Segraves K A, Kristeller J L, Xu W C (1999) Antidepressant pharmacotherapy helps some cigarette smokers more than others. Journal of Consulting & Clinical Psychology. 67: 547-554.
Hughes J R, Stead L F, Lancaster T (2005) Nortriptyline for smoking cessation: a review. Nicotine & Tobacco Research 7: 491-9.
James W P, Caterson I D, Coutinho W, Finer N, Van Gaal L F, Maggioni A P, Torp-Pedersen C, Sharma A M, Shepherd G M, Rode R A, Renz C L. Effect of sibutramine on cardiovascular outcomes in overweight and obese subjects. N Engl J Med. 2010, 363:905-17.
Johnson J E, Slade S, Wells C, Petro A, Sexton H, Rezvani A H, Brown M L, Paige M A, McDowell B E, Xiao Y, Kellar K J, Levin E D (2012) Chronic sazetidine-a infusion reduces nicotine self-administration in both male and female rats. Psychopharmacology under review
Kokkinidis L, Zacharko R M, Anisman H. Amphetamine withdrawal: a behavioral evaluation. Life Sci. 1986 Apr. 28; 38(17):1617-23.
Koob G F. Neurobiology of addiction. Toward the development of new therapies. Ann N Y Acad Sci. 2000; 909:170-85.
Lê A D, Li Z, Funk D, Shram M, Li T K, Shaham Y. Increased vulnerability to nicotine self-administration and relapse in alcohol-naive offspring of rats selectively bred for high alcohol intake. J Neurosci. 2006 Feb. 8; 26(6):1872-9.
Lengyel K, Pieschl R, Strong T, Molski T, Mattson G, Lodge N J, Li Y W (2008) Ex vivo assessment of binding site occupancy of monoamine reuptake inhibitors: methodology and biological significance. Neuropharmacology 55: 63-70.
Levin E D, Johnson J, Slade S, Wells C, Cauley M, Petro A, Rose J E (2011) Lorcaserin decreases nicotine self-administration in female rats. Journal of Pharmacology and Experimental Therapeutics 338: 890-896.
Levin E D, Westman E C, Stein R M, Carnahan E, Sanchez M, Herman S, Behm F M, Rose J E (1994) Nicotine skin patch treatment increases abstinence, decreases withdrawal symptoms and attenuates rewarding effects of smoking. Journal of Clinical Psychopharmacology 14: 41-49.
Li X, Rainnie D G, McCarley R W, Greene R W (1998) Presynaptic nicotinic receptors facilitate monoaminergic transmission. Journal of Neuroscience. 18: 1904-12.
Lüscher C, Ungless M A (2006) The Mechanistic Classification of Addictive Drugs. PLoS Med 3(11): e437. doi:10.1371/journal.pmed.0030437
Majchrowicz, E. (1973), Alcohol, aldehydes, and biogenic amines. Annals of the New York Academy of Sciences, 215: 84-88. doi: 10.1111/j.1749-6632.1973.tb28252.x.
Markou A, Kosten T R, Koob G F. Neurobiological similarities in depression and drug dependence: a self-medication hypothesis. Neuropsychopharmacology. 1998 March; 18(3):135-74.
Miller N S, Goldsmith R J. Craving for alcohol and drugs in animals and humans: biology and behavior. J Addict Dis. 2001; 20(3):87-104.
Montgomery S A, Åsberg M. A new depression scale designed to be sensitive to change. Br J Psychiatry 1979; 134:382-9
Montgomery, S. A. & Åsberg, M. (1979) A New Depression Scale Designed To Be Sensitive To Change. British Journal of Psychiatry. Vol. 134, pp. 382-389.
Noda Y, Kamei H, Mamiya T, Furukawa H, Nabeshima T. Repeated phencyclidine treatment induces negative symptom-like behavior in forced swimming test in mice: imbalance of prefrontal serotonergic and dopaminergic functions. Neuropsychopharmacology. 2000 October; 23(4):375-87.
Olsen, L. R., et al., The internal and external validity of the Major Depression Inventory in measuring severity of depressive states Psychological Medicine (2003), 33: 351-356 Cambridge University Press.
Paterson N E, Markou A. Animal models and treatments for addiction and depression comorbidity. Neurotox Res. 2007 January; 11(1):1-32.
Pierce R C, Kalivas P W. A circuitry model of the expression of behavioral sensitization to amphetamine-like psychostimulants. Brain Res Brain Res Rev. 1997 October; 25(2):192-216.
Pliakas A M, Carlson R R, Neve R L, Konradi C, Nestler E J, Carlezon W A Jr. Altered responsiveness to cocaine and increased immobility in the forced swim test associated with elevated cAMP response element-binding protein expression in nucleus accumbens. J Neurosci. 2001 Sep. 15; 21(18):7397-403.
Rascol O, Poewe W, Lees A, Aristin M, Salin L, Juhel N, Waldhause L, Schindler T. Tesofensine (NS2330), a Monoamine Reuptake Inhibitor, in Patients with Advanced Parkinson Disease and Motor Fluctuations. Arch. Neurol 2008, 65:577-583.
Rezvani A H, Overstreet D H, Janowsky D S (1990) Genetic serotonin deficiency and alcohol preference in the Fawn-Hooded rats. Alcohol and Alcoholism 25: 573-575.
Schroeder B E, Binzak J M, Kelley A E. A common profile of prefrontal cortical activation following exposure to nicotine- or chocolate-associated contextual cues. Neuroscience. 2001; 105(3):535-45.
Sepracor Press Release, Jul. 1, 2009 http://www.fiercebiotech.com/press-releases/sepracor-provides-update-clinical-trials-sep-225289-and-lunesta-r-pediatrics.
Sharples C G, Kaiser S, Soliakov L, Marks M J, Collins A C, Washburn M, Wright E, Spencer J A, Gallagher T, Whiteaker P, Wonnacott S (2000) UB-165: a novel nicotinic agonist with subtype selectivity implicates the alpha4beta2* subtype in the modulation of dopamine release from rat striatal synaptosomes. Journal of Neuroscience 20: 2783-2791.
Sharples C G, Kaiser S, Soliakov L, Marks M J, Collins A C, Washburn M, Wright E, Spencer J A, Gallagher T, Whiteaker P, Wonnacott S. UB-165: a novel nicotinic agonist with subtype selectivity implicates the alpha4beta2* subtype in the modulation of dopamine release from rat striatal synaptosomes. J Neurosci. 2000 Apr. 15; 20(8):2783-91.
Shiffman S, Johnston J A, Khayrallah M, Elash C A, Gwaltney C J, Paty J A, Gnys M, Evoniuk G, DeVeaugh-Geiss J (2000) The effect of bupropion on nicotine craving and withdrawal. Psychopharmacology 148: 33-40.
Skolnick M, Popik P, Janowsky A, Beer B, Lippa A: Antidepressant-like actions of DOV 21,947: A triple reuptake inhibitor. European Journal of Pharmacology 2003 461:99-104
Skolnick P, Popik P, Janowsky A, Beer B, Lippa A S (2003) Antidepressant-like actions of DOV 21,947: a “triple” reuptake inhibitor. European Journal of Pharmacology 461: 99-104.
Stoker A K, Semenova S, Markou A (2008) Affective and somatic aspects of spontaneous and precipitated nicotine withdrawal in C57BL/6J and BALB/cByJ mice. Neuropharmacology 54: 1223-1232.
Tizzano J P, Stribling D S, Perez-Tilve D, Strack A, Frassetto A, Chen R Z, Fong T M, Shearman L, Krieter P A, Tschop M H, Skolnick P, Basile A S (2008) The triple uptake inhibitor (1R,5S)-(+)-1-(3,4-dichlorophenyl)-3-azabicyclo[3.1.0]hexane hydrochloride (DOV 21947) reduces body weight and plasma triglycerides in rodent models of diet-induced obesity. Journal of Pharmacology and Experimental Therapeutics 324: 1111-1126.
Tran P, Skolnick P, Czobor P, Huang N Y, Bradshaw M, McKinney A, Fava M (2012) Efficacy and tolerability of the novel triple reuptake inhibitor amitifadine in the treatment of patients with major depressive disorder: A randomized, double-blind, placebo-controlled trial. Journal of Psychiatric Research 46: 64-71.
Trivedi M H, Rush A J, Ibrahim H M et al. The Inventory of Depressive Symptomatology, Clinician Rating (IDS-C) and Self-Report (IDS-SR), and the Quick Inventory of Depressive Symptomatology, Clinician Rating (QIDS-C) and Self-Report (QIDS-SR) in public sector patients with mood disorders: a psychometric evaluation. Psychol Med 2004; 34(1):73-82.
Warren, W. L. Revised Hamilton Rating Scale for Depression (RHSD) (1994) Los Angeles, Western Psychological Services.
Wilens T E, Klint T, Adler L, West S, Wesnes K, Graff O, Mikkelsen B. A Randomized Controlled Trial of a Novel Mixed Monamine Reuptake Inhibitor in Adults with ADHD. Behavioral and Brain Functions 2008, 4:24.

1-21. (canceled)

22. A method for treating a nicotine-related disorder in a human subject in need thereof, comprising administering to the subject 100 to 250 mg of (+)-1-(3,4-dichlorophenyl)-3-azabicyclo[3.1.0]hexane or a pharmaceutically acceptable salt thereof substantially free of (−)-1-(3,4-dichlorophenyl)-3-azabicyclo[3.1.0]hexane or a pharmaceutically acceptable salt thereof.

23. The method according to claim 22, wherein the (+)-1-(3,4-dichlorophenyl)-3-azabicyclo[3.1.0]hexane or pharmaceutically acceptable salt thereof has no more than about 2% w/w of (−)-1-(3,4-dichlorophenyl)-3-azabicyclo[3.1.0]hexane or pharmaceutically acceptable salt thereof.

24. The method according to claim 22, wherein the (+)-1-(3,4-dichlorophenyl)-3-azabicyclo[3.1.0]hexane or pharmaceutically acceptable salt thereof has no more than about 1% w/w of (−)-1-(3,4-dichlorophenyl)-3-azabicyclo[3.1.0]hexane or pharmaceutically acceptable salt thereof.

25. The method according to claim 22, wherein the nicotine-related disorder is selected from the group consisting of Nicotine Dependence, Nicotine Withdrawal, Nicotine Cessation, Nicotine Relapse, and Nicotine-Related Disorder not otherwise specified (NOS).

26. The method according to claim 22 further comprising coordinately administering a secondary therapeutic agent.

27. The method according to claim 26, wherein the secondary therapeutic agent is an anti-nicotine agent.

28. The method according to claim 27, wherein the anti-nicotine agent is selected from the group consisting of varenicline, bupropion, cytisine, anabasine, nortriptyline, mecamylamine, and clonidine.

29. The method according to claim 26, wherein the subject is effectively treated for a secondary, co-morbid central nervous system (CNS) condition or addictive disorder selected from the group consisting of depression, anxiety, and psychosis.

30. The method according to claim 26, wherein the secondary therapeutic agent is an anti-depressant.

31. The method according to claim 26, wherein the secondary therapeutic agent is an anti-psychotic drug.

32. The method according to claim 26, wherein the secondary therapeutic agent is an anxiolytic agent.

33. A method for treating nicotine consumption or addiction comprising administering to a patient in need thereof 100 to 250 mg of (+)-1-(3,4-dichlorophenyl)-3-azabicyclo[3.1.0]hexane or a pharmaceutically acceptable salt thereof substantially free of (−)-1-(3,4-dichlorophenyl)-3-azabicyclo[3.1.0]hexane or a pharmaceutically acceptable salt thereof.

34. The method according to claim 33, wherein the (+)-1-(3,4-dichlorophenyl)-3-azabicyclo[3.1.0]hexane or pharmaceutically acceptable salt thereof has no more than about 2% w/w of (−)-1-(3,4-dichlorophenyl)-3-azabicyclo[3.1.0]hexane or pharmaceutically acceptable salt thereof.

35. The method according to claim 33, wherein the (+)-1-(3,4-dichlorophenyl)-3-azabicyclo[3.1.0]hexane or pharmaceutically acceptable salt thereof has no more than about 1% w/w of (−)-1-(3,4-dichlorophenyl)-3-azabicyclo[3.1.0]hexane or pharmaceutically acceptable salt thereof.

36. The method according to claim 33 further comprising coordinately administering a secondary therapeutic agent.

37. The method according to claim 36, wherein the secondary therapeutic agent is an anti-nicotine agent.

38. The method according to claim 37, wherein the anti-nicotine agent is selected from the group consisting of varenicline, bupropion, cytisine, anabasine, nortriptyline, mecamylamine, and clonidine.

39. The method according to claim 22, wherein the effective amount of (+)-1-(3,4-dichlorophenyl)-3-azabicyclo[3.1.0]hexane or pharmaceutically acceptable salt thereof substantially free of (−)-1-(3,4-dichlorophenyl)-3-azabicyclo[3.1.0]hexane or pharmaceutically acceptable salt thereof is administered in a sustained release formulation.
